(19) United States
(12) Patent Application Publication
(10) Pub. No.: US 2019/0307893 A1
(43) Pub. Date: Oct. 10, 2019

(54) NANOVESICLES WITH PORPHYRIN-LIPID CONJUGATE CORE

(71) Applicant: UNIVERSITY HEALTH NETWORK, Toronto (CA)

(72) Inventors: Gang Zheng, Toronto (CA); Kenneth Ng, Toronto (CA); Juan Chen, Toronto (CA); Misa Takada, Osaka (JP); Kara Harmatys, Toronto (CA)

(21) Appl. No.: 16/086,235

(22) PCT Filed: Mar. 10, 2017
(86) PCT No.: PCT/CA2017/000052

§ 371 Date: Sep. 18, 2018

Related U.S. Application Data
(60) Provisional application No. 62/310,108, filed on Mar. 18, 2016.

(51) Int. Cl. A61K 47/69; A61K 47/54; A61K 9/127; C07K 17/02; A61K 41/00

(52) U.S. Cl. CPC A61K 47/6915; A61K 47/544; A61K 9/1271; A61K 41/0071; C07K 17/02; A61K 47/546; A61K 47/6917

(57) ABSTRACT

The application relates to liposomal nanovesicles comprising porphyrin-lipid conjugates within the liposomal lipid bilayer. Said porphyrin-lipid conjugate comprise porphyrins that are modified with a —CH(R¹)—O—R² group and that chelate a metal ion. Such modifications of the porphyrin allow for ordered assembly in the lipid bilayer of the nanovesicles while resulting in a bathochromic shift in the wavelength of light absorbed by the porphyrin chromophore. These nanovesicles can be used for photothermal therapy, photodynamic therapy, photoacoustic imaging and fluorescence imaging. The application also teaches methods for preparing the porphyrin-lipid conjugates and the nanovesicles.

### FIELD OF THE INVENTION

The invention relates to the field of nanovesicles and, more specifically, to monolayer nanovesicles having porphyrin-lipid conjugates in the hydrophobic core.

### BACKGROUND OF THE INVENTION

Green photosynthetic bacteria are phototrophs that thrive in dimly light environments and possess one of the most efficient light harvesting systems characterized in any photosynthetic organism. These microbes reside at depths of 100 m under the sea surface with some species discovered at greater depths, near underwater near-infrared (NIR) light-emitting thermal vents.^{1, 2} The ability for these microbes to achieve a high photosynthetic efficiency can be attributed to their remarkable light harvesting organelles known as the chlorosome. Chlorosomes are flattened, ellipsoidal, lipid-encapsulated structures that contain a three-dimensional supramolecular assembly of coherently coupled bacteriochlorophyll (Bchl) molecules (Bchl c/d/e).³⁻⁵ Unlike the light harvesting centers of other phototrophs, intermolecular interactions between chromophores are dictated by the ordered packing of dyes that occur in the absence of a structural protein scaffold.^{5, 6} The strong and coherent coupling of BchIs⁶ enables long range transport of absorbed light energy as well as a NIR-shifted increase in optical absorption of its S₀→S₁ transition. Several models have been proposed for the geometric arrangement of Bchl c in chlorosomes that are responsible for the shifted absorption. While the specific bonds involved in the interactions for each model differ, all models cite the importance of the centrally coordinated metal atom, capable of forming coordination bonds with nucleophilic substituents located around the Bchl c ring in adjacent molecules.⁷⁻⁹

Synthetic and semi-synthetic Bchl mimics have been constructed in order to elucidate the structural requirements for the optical properties which arise from the self-assembly of the dyes. Past research on modifying naturally-derived chlorin molecules have shown that several key conversions in its structure can promote self-assembly of the dyes into ordered aggregates. These include the insertion of a central metal atom capable of forming pentacoordinated bonds (four with the porphyrin and one with an axial ligand)¹⁰, and modification of the 3¹-vinyl sidegroup with either a hydroxyl^{6, 11, 12} or a methoxy substitution.^{12,13} The oxygen atom helps maintain a slipped interchromophore packing arrangement by acting as an axial ligand for the centrally coordinated metal. In the case of 3′-hydroxy, additional hydrogen bonding interactions with oxygen at the 13¹-position carbonyl group enables the formation of planar aggregate arrangements.

It is advantageous to investigate these systems, as the implications arising from their study could lead to the development of more efficient dye-based light capturing agents for solar energy harvesting. Beyond advancing research in biomimetic solar harvesting technologies, the ability to generate these ordered structures with red-shifted and intense optical absorption cross-sections may also have medical applications such as photoacoustic (PA) imaging¹⁴, fluorescence imaging¹⁵, and photothermal therapy. One challenge in working with these ordered aggregate systems is to promote organized self-assembly, while maintaining solubility and controlling the size of the assemblies. Early work by Miyatake et al., showed that zinc metallochlorins can form self-assembled aggregates in surfactant systems such as Triton X-100 and α-lecithin.¹⁶ These aggregates were thought to partition to the hydrophobic core of the surfactant micelles due to the fact that the altered spectra from aggregation occurred at surfactant concentrations, which closely corresponded with the critical micelle concentration.¹⁶ Despite these advances, there is merit in constructing metallochlorins that can form ordered aggregates in other types of lipid nanostructures such as bilayer nanovesicles. Firstly, the rigid and aligned environment of phospholipid bilayers can act as a scaffold to facilitate the binding and assembly of the metallochlorin dyes. Secondly, inducing ordered aggregation in the bilayer membrane, frees the hydrophilic core for loading of various aqueous soluble payloads.^{17, 18} Lastly, phase-sensitive membranes can potentially induce or inhibit aggregate formation; enabling the creation of stimuli-responsive optical materials¹⁴ and novel supramolecular contrast agents for non-linear third harmonic generation microscopy¹⁹. Successful formation of these nanoscale assemblies in aligned lipid environments using these metal coordination techniques will likely expand the application of these supramolecular assemblies for phototheranostic applications.²⁰

### SUMMARY OF INVENTION

According to one aspect, there is provided a monolayer nanovesicle with a hydrophobic core is prepared. The monolayer comprises phospholipids and the hydrophobic core contains porphyrin-lipid conjugates. The porphyrin-lipid conjugate is comprised of two main components: 1) a porphyrin, porphyrin derivative or porphyrin analog, and 2) a lipid covalently bonded to the porphyrin, porphyrin derivative or porphyrin analog. The lipid is an unsaturated or branched fatty acid that anchors the porphyrin-lipid conjugate to the monolayer. The porphyrin, porphyrin derivative or porphyrin analog is in turn comprised of two main elements: a) a CH(R¹)—O—R² group covalently bonded to a carbon on a porphyrin ring of the porphyrin, porphyrin derivative or porphyrin analog, wherein R¹ and R² are independently H or a C₁₋₄ alkane; and b) a metal chelated in the porphyrin, porphyrin derivative or porphyrin analog.

According to a further aspect, there is provided a method of monitoring delivery of a nanovesicle to a target area in a subject comprising providing the nanovesicle described herein; administering the nanovesicle to the subject; and monitoring the progress of the nanovesicle to the target area by irradiating with a wavelength of light, preferably in the form a pulsed beam, wherein the nanovesicle emits a photoacoustic signal in response to the wavelength of light, and measuring the photoacoustic signal in the subject.

According to a further aspect, there is provided a method of performing photothermal therapy on a target area in a subject comprising providing the nanovesicle described herein; administering the nanovesicle to the subject; and irradiating the nanovesicle at the target area with a wavelength of light, wherein the wavelength of light increases the temperature of nanovesicle.

According to a further aspect, there is provided a method of imaging a target area in a subject, comprising providing the nanovesicle described herein; administering the nanovesicle to the subject; irradiating the nanovesicle at the target area with a wavelength of light, wherein the nanovesicle emits a photoacoustic signal in response to the wavelength of light; and measuring and/or detecting the photoacoustic signal at the target area.

According to a further aspect, there is provided a method of imaging a target area in a subject, comprising providing the nanovesicle described herein; administering the nanovesicle to the subject; and measuring and/or detecting the fluorescence at the target area.

According to a further aspect, there is provided a method of performing photodynamic therapy at a target area in a subject, comprising providing the nanovesicle described herein; administering the nanovesicle to the subject; and allowing the porphyrin-lipid conjugate to disassociate from the nanovesicle at the target area; and irradiating the target area with a wavelength of light, wherein the wavelength of light activates the nanovesicle to generate singlet oxygen.

According to a further aspect, there is provided a method comprising a combination of any of the methods described herein.

According to a further aspect, there is provided a use of the nanovesicle described herein for performing photodynamic therapy.

According to a further aspect, there is provided a use of the nanovesicle described herein for performing photothermal therapy.

According to a further aspect, there is provided a use of the nanovesicle described herein for performing photoacoustic imaging.

According to a further aspect, there is provided a use of the nanovesicle described herein for performing fluorescence imaging.

### BRIEF DESCRIPTION OF FIGURES

Embodiments of the invention may best be understood by referring to the following description and accompanying drawings. In the drawings:

FIG. 1 shows a schematic of zinc chlorin lipid molecules templated within the membrane of lipid nanovesicles. The presence of the 3¹-methoxy group and inserted zinc atom enables formation of metal coordination bonds responsible for ordered assembly of the dye.

FIG. 2 shows generalized structure for series of chlorin molecules studied (top). Data showing the identity of the R groups for each compound and the UV/visible absorption maximum for the dye's Qy absorption band in methanol or embedded within nanovesicle membranes (bottom).

FIG. 3 shows the effect of lipid conjugation on the incorporation of zinc chlorin derivatives into lipid nanovesicles. (A) Ratio of Zn-MeO-chlorin acid, Zn-MeO-chlorin lipid to total lipids used in formulation. For each sample, initial 1, 10 or 20 chlorin dye loading (mol/mol % of total dye and lipid percentage) was applied. Each bar represents the mean±S.D. of 3 independent measurements. (B) Photographs of Zn-MeO-chlorin acid (20%) and Zn-MeO-chlorin lipid (20%) after freeze and thaw cycles (day 0) and after storage at room temperature for 10 d. Precipitate was observed for the Zn-MeO-chlorin acid samples after 10 d of storage.

FIG. 4 shows UV/Visible absorption and circular dichroism (CD) spectra for each of four compounds embedded in lipid nanovesicles in the intact (black solid) and detergent-disrupted (red dashed) state. (A) 1% Zn-MeO chlorin acid, (B) 20% Zn-vinyl-chlorin lipid, (C) 20% MeO-chlorin lipid and (D) 20% Zn-MeO-chlorin lipid.

FIG. 5 shows influence of Zn-MeO-chlorin lipid loading ratio on absorption red-shift. The amount of Zn-MeO-chlorin lipid (mol %) added to the total amount of phospholipid was varied from 1-30 mol %. All samples were maintained at the sample concentration. Dotted line represents 0.1% triton X-100 detergent disrupted control samples. (A) UV/Visible traces display red-shift and increased absorption as the loading of Zn-MeO-chlorin lipid was increased. (B) Plot of the normalized absorption (from A) at 725 nm (blue) and 661 nm (red). Each point represents the average±S.D. of three independent samples. (C) CD of Zn-MeO-chlorin lipid nanovesicles at various loading ratios.

FIG. 6 shows (A) PA spectra of Zn-MeO-chlorin lipid nanovesicles in intact (black solid) and disrupted (red dashed) state. (B) Concentration dependence of PA signal (725 nm, 21 MHz). Each lane represents PBS (i), Zn-MeO-chlorin lipid (653 nm in MeOH) at 1.7 O.D. (ii), 3.3 O.D. (iii), 5.0 O.D. (iv), and 6.6 O.D. (v). (C) Plot of PA signal as a function of monomeric optical absorption of samples containing nanovesicles that are intact (black) or disrupted (red) with 0.1% Triton X-100 detergent. Each data point represents the mean±S.D. of 3 measurements. (D) Representative PA image of hamster cheek pouch tumor prior to injection (top) and 5 min after intravenous administration of Zn-MeO-chlorin lipid nanovesicles (bottom). The scale bar represents 2 mm. (E) Average PA spectrum of tumor before (red) and 5 min after (black) Zn-MeO-chlorin lipid nanovesicle injection.

FIG. 7 shows the effect of lipid conjugation on the incorporation of Zn-vinyl chlorin lipid (20%) and MeO-chlorin lipid (20%) compounds into lipid nanovesicles. Dye recovery after extrusion in 20% chlorin lipid nanovesicle samples prepared using MeO-chlorin lipid (red) and Zn-vinyl-chlorin lipid (black).

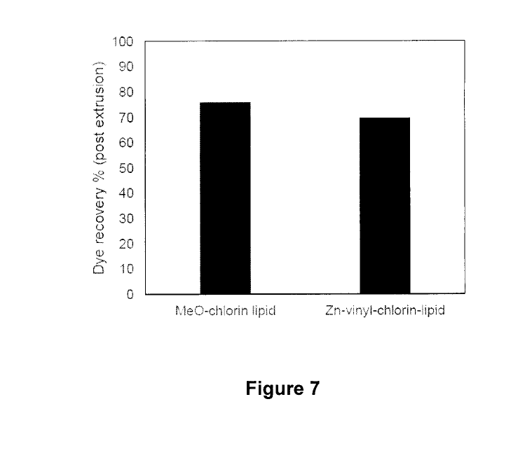

FIG. 8 shows fluorescence emission of 20 mol % Zn-MeO-chlorin lipid doped in lipid nanovesicles. Fluorescence emission from samples in the intact (black solid) and detergent-disrupted (0.1% Triton X-100) (red dashed) states demonstrating the structurally driven fluorescence quenching properties.

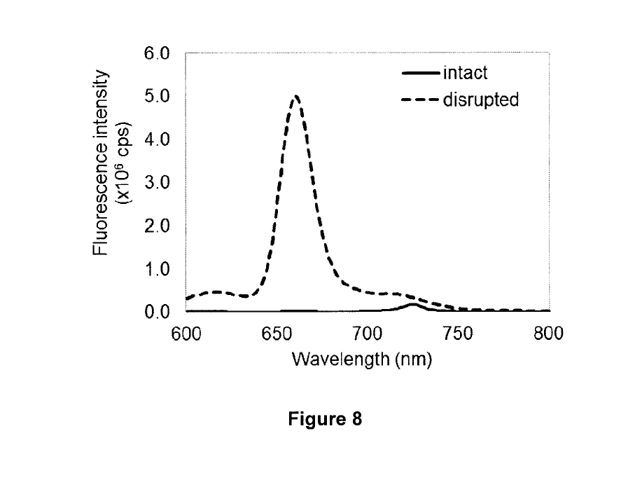

FIG. 9 shows a schematic of zinc chlorin oleate molecules assembled within the core of a lipoprotein nanoparticle. The presence of the 3¹-methoxy or 3¹-hydroxy group and inserted zinc atom enables formation of metal coordination bonds responsible for ordered assembly of the dye.

FIG. 10 shows a generalized structure for series of chlorin molecules studied in this report (top). Table indicating the identity of the R groups for each compound (bottom).

FIG. 11 shows UV-Vis absorbance (A, C) and mass spectrograph (B, D) of 13²-demethoxycarbonylpheophorbide-a oleylamine (Vinyl-chlorin oleylamine, 3) and Zinc 13²-demethoxycarbonylpheophorbide-a oleylamine (Zn-vinyl-chlorin oleylamine, 4), respectively.

FIG. 12 shows UV-Vis absorbance (A, C) and mass spectrograph (B, D) of 3-Devinyl-3¹-methoxymethyl-13²-demethoxycarbonylpheophorbide-a oleate (MeO-chlorin oleylamine) (16) and Zinc 3-Devinyl-3¹-methoxymethyl-13²-demethoxycarbonylpheophorbide-a oleate (Zn-MeO-chlorin oleylamine) (17), respectively.

FIG. 13 shows UV-Vis absorbance (A, C) and mass spectrograph (B, D) of 3-Devinyl-3¹-hydroxyl-13²-demethoxycarbonylpheophorbide-a oleylamine (OH-chlorin oleylamine) (9) and Zinc 3-Devinyl-3¹-hydroxyl-13²-demethoxycarbonylpheophorbide-a oleylamine (Zn—OH-chlorin oleylamine) (10), respectively.

FIG. 14 showed the absorption of different Zn-chlorin oleylamine alternate when loaded in lipoprotein nanoparticles (e.g. Zn-vinyl-chlorin oleylamine, Zn-MeO-chlorin oleylamine, Zn—OH-chlorin oleylamine) and the corresponding absorption when the nanostructures were disrupted. The Zn—OH-chlorin oleylamine loaded nanoparticles showed distinguished absorption shift from 654 nm to 715 nm.

FIG. 15 shows (A) PA spectra of Zn—OH-chlorin oleylamine lipoprotein nanoparticles in intact (red solid) and disrupted (red dashed) state. (B) Concentration dependence of PA spectral signal (PBS, 10 μM, 20 μM, 40 μM and 80 μM). (C) PA signal of indicated concentrations for intact and disrupted (80 μM) lipoprotein nanoparticles at 715 nm (21 MHz).

FIG. 16 shows a schematic of a porphyrin nanoparticle with chlorosome-like assembly.

### DETAILED DESCRIPTION

Chlorosomes are vesicular light-harvesting organelles found in photosynthetic green sulfur bacteria. These organisms thrive in low photon flux environments due to the most efficient light-to-chemical energy conversion, promoted by a protein-less assembly of chlorin pigments. These assemblies possess collective absorption properties and can be adapted for contrast-enhanced bioimaging applications, where maximized light absorption in the near-infrared optical window is desired. Light absorption can be tuned towards the near-infrared region by engineering a chlorosome-inspired assembly of synthetic metallochlorins in a biocompatible lipid scaffold. In a series of synthesized chlorin analogues, it was discovered that lipid-conjugation, central coordination of a zinc metal into the chlorin ring and a 3′-methoxy or 3¹-hydroxy substitution were critical for the formation of dye assemblies in lipid nanovesicles. The substitutions result in a specific optical shift, characterized by a bathochromically-shifted (74 nm) Q_{y} absorption band, along with an increase in absorbance and circular dichroism as the ratio of dye-conjugated lipid was increased. These alterations in optical spectra are indicative of the formation of delocalized excitons states across each molecular assembly. This strategy of tuning absorption by mimicking the structures found in photosynthetic organisms may spur new opportunities in the development of biophotonic contrast-agents for medical applications.

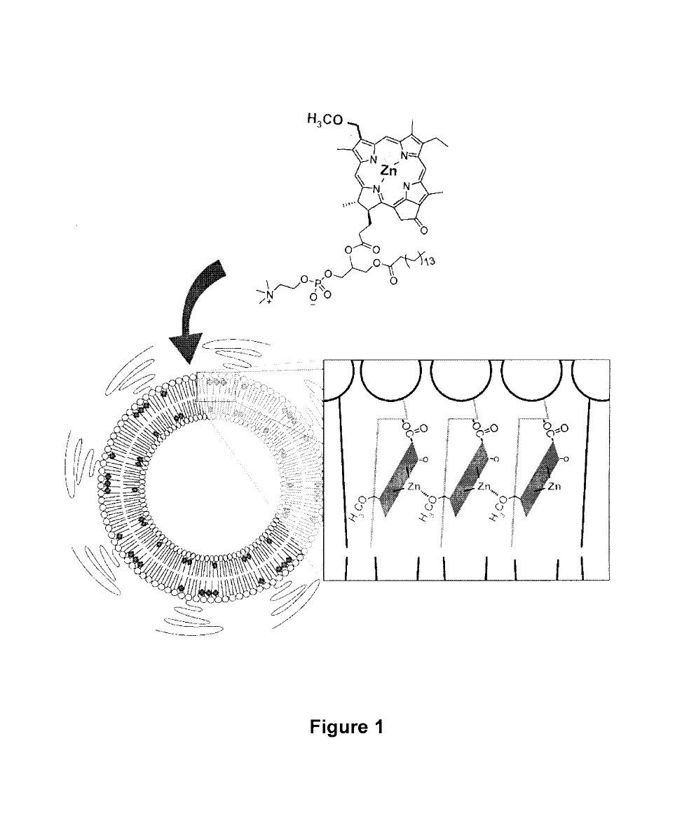

Turning to FIG. 1, lipid-conjugated chlorin derivatives were examined to see whether they can be induced to form ordered assemblies in bilayer lipid nanovesicles by making chemical modifications to the chlorin ring with the goal of facilitating ordered intermolecular interactions between the chlorin dyes. Furthermore, liposomes were investigated for their ability to stabilize the aggregate structure and examine their application as contrast agents for PA imaging.

According to one aspect, there is provided a monolayer nanovesicle with a hydrophobic core is prepared. The monolayer comprises phospholipids and the hydrophobic core contains porphyrin-lipid conjugates. The porphyrin-lipid conjugate is comprised of two main components: 1) a porphyrin, porphyrin derivative or porphyrin analog, and 2) a lipid covalently bonded to the porphyrin, porphyrin derivative or porphyrin analog. The lipid is an unsaturated or branched fatty acid that anchors the porphyrin-lipid conjugate to the monolayer. The porphyrin, porphyrin derivative or porphyrin analog is in turn comprised of two main elements: a) a CH(R¹)—O—R² group covalently bonded to a carbon on a porphyrin ring of the porphyrin, porphyrin derivative or porphyrin analog, wherein R¹ and R² are independently H or a C₁₋₄ alkane; and b) a metal chelated in the porphyrin, porphyrin derivative or porphyrin analog.

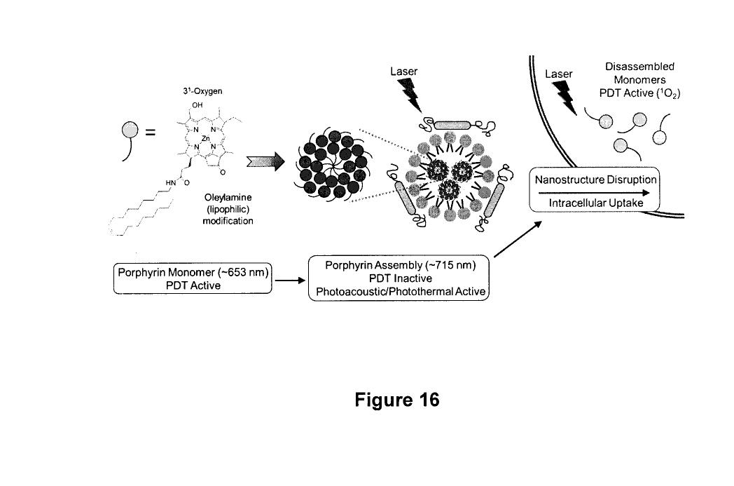

An example of the monolayer nanovesicle described above is illustrated schematically in FIG. 16.

1) Porphyrin, Porphyrin Derivative or Porphyrin Analog

Porphyrins are a group of heterocyclic macrocycle organic compounds containing a porphyrin ring. As used herein, “porphyrin ring” refers to a chemical structure composed of four modified pyrrole subunits interconnected at their a carbon atoms via methine bridges (═CH—), as illustrated in Formula 1 below. The carbon atoms of the porphyrin ring can be substituted at various locations, which are well known in the art.

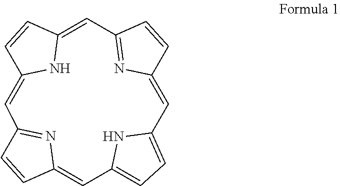 [SMILES: C1=Cc2cc3ccc(cc4nc(cc5ccc(cc1n2)[nH]5)C=C4)[nH]3]

“Pyrrole rings” as used herein refer to the four modified pyrrole subunits of the porphyrin ring.

Exemplary porphyrin, porphyrin derivative or porphyrin analog of the porphyrin-lipid conjugate includes but are not limited to hematoporphyrins, protoporphyrins, tetraphenylporphyrins, pyropheophorbides, bacteriochlorophylls, chlorophyll a, benzoporphyrin derivativs, tetrahydroxyphenyl chlorins, purpurins, benzochlorins, naphthochlorins, verdins, rhodins, keto chlorins, azachlorins, bacteriochlorins, tolyporphyrins, benzobacteriochlorins, expanded porphyrins (such as texaphyrins, sapphyrins, and hexaphyrins) and porphyrin isomers (such as porphycenes, inverted porphyrins, phthalocyanines, and naphthalocyanines). In preferred embodiments, the porphyrin, porphyrin derivative or porphyrin analog is pyropheophorbide-a chlorin.

In some embodiments the lipid and the CH(R¹)—O—R² group are bonded to separate pyrrole rings on the porphyrin ring. In preferred embodiments, the lipid and the CH(R¹)—O—R² group are bonded to adjacent pyrrole rings on the porphyrin ring. For example, the CH(R¹)—O—R² group may be bonded to the carbon at position 3 of the porphyrin ring.

a) CH(R¹)—O—R² Group

A CH(R¹)—O—R² group is covalently bonded to a carbon on a porphyrin ring of the porphyrin, porphyrin derivative or porphyrin analog. R¹ and R² are independently H or a C₁₋₄ alkane.

In preferred embodiments, R¹ and R² are independently methyl or ethyl, and more preferably both methyl.

b) Chelated Metal

Chelation of metals in a porphyrin, porphyrin derivative or porphyrin analog has been described in the art. In some embodiments, the metal chelated in the porphyrin, porphyrin derivative or porphyrin analog is Mg, Mn, Fe, Ni, Zn, Cu, Co or Pd. Preferably, the metal is Fe, Zn, Cu, Co, or Pd, and more preferably the metal is Zn or Pd.

c) Ketone Group

Optionally, the porphyrin, porphyrin derivative or porphyrin analog further comprises c) a ketone group bonded to a carbon on the porphyrin ring.

In preferred embodiments, this ketone group is bonded to a pyrrole ring opposite to the pyrrole ring bonded to the CH(R¹)—O—R² group. For example, the ketone group may be bonded to the carbon at position 13 of the porphyrin ring.

2) Lipid

A lipid is covalently bonded to the porphyrin, porphyrin derivative or porphyrin analog. The lipid is an unsaturated or branched fatty acid that anchors the porphyrin-lipid conjugate to the monolayer.

In some embodiments, the lipid fatty acid comprises at least one oleate/oleylamine moiety, cholesterol oleate moiety, farnesyl moiety or phytol moiety. In preferred embodiments, the fatty acid is oleylamine.

Other types of fatty acids that can be used to anchor the porphyrin-lipid conjugate to the monolayer are well known in the art, and are described in “Reconstitution of the hydrophobic core of low-density lipoprotein”, Krieger M., Methods Enzymol. 1986; 128:608-13.

In some embodiments, the lipid fatty acid is bonded to the carbon at position 7, 10, 17 or 20 of the porphyrin ring. In preferred embodiments, the fatty acid is bonded to the carbon at position 17 of the porphyrin ring.

Phospholipid Monolayer

Turning now to the monolayer, the monolayer is comprised of phospholipid.

As used herein, “phospholipid” is a class of lipids having a hydrophilic head group comprised of a phosphate group, and hydrophobic lipid tail(s) joined together by a glycerol molecule. Because of their amphiphilic characteristic, they can form, among others, lipid bilayers.

Examples of the phospholipid include but are not limited to phosphatidylcholine, phosphatidylethanoloamine, phosphatidylserine, phosphatidic acid, phosphatidylglycerols, phosphatidylinositol or a combination thereof. Exemplary phospholipids include 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)](DPPG) or combinations thereof.

In some embodiments, the monolayer comprises at least one peptide incorporated therein. The peptide comprises an amino acid sequence capable of forming at least one amphipathic α-helix. Exemplary peptides include Class A, H, L and M amphipathic α-helices and fragments thereof, or peptides comprising a reversed peptide sequence of said Class A, H, L and M amphipathic α-helices and fragments thereof.

In some embodiments, the peptide is a peptide of an apolipoprotein or an apolipoprotein mimetic. Examples of apolipoproteins and apolipoprotein mimetics are well known in the art, and are described for example in U.S. Pat. No. 6,329,341 B1, as well as in references such as THE JOURNAL OF BIOLOGICAL CHEMISTRY, 1989, 264, pages 4628-4635.

In other embodiments, examples of the peptide are disclosed in PCT publication no. WO 2009/073984.

In some embodiments, the nanovesicle further comprises a PEG phospholipid, preferably DPPE-mPEG2000.

In some embodiments, the molar % of porphyrin-lipid conjugate to phospholipid is up to 70%. Preferably, the molar % is between 1% to 50%. More preferably, the molar % is about 30%, and even more preferably about 20%.

The Nanovesicle

In some embodiments, the nanovesicle is substantially spherical and about 10-50 nm in diameter.

In some embodiments, the nanovesicle exhibits a bathochromic shift towards the infrared spectrum. Preferably, the bathochromic shift is 40 nm-80 nm.

In some embodiments, the nanovesicle further comprises a targeting molecule. “Targeting molecule” is any molecule that can direct the nanovesicle to a particular target, for example, by binding to a receptor or other molecule on the surface of a targeted cell. Targeting molecules may be proteins, peptides, nucleic acid molecules, saccharides or polysaccharides, receptor ligands or other small molecules. The degree of specificity can be modulated through the selection of the targeting molecule. For example, antibodies typically exhibit high specificity. These can be polyclonal, monoclonal, fragments, recombinant, or single chain, many of which are commercially available or readily obtained using standard techniques.

Therapeutic Uses

According to a further aspect, there is provided a method of monitoring delivery of a nanovesicle to a target area in a subject comprising providing the nanovesicle described herein; administering the nanovesicle to the subject; and monitoring the progress of the nanovesicle to the target area by irradiating with a wavelength of light, preferably in the form a pulsed beam, wherein the nanovesicle emits a photoacoustic signal in response to the wavelength of light, and measuring the photoacoustic signal in the subject.

According to a further aspect, there is provided a method of performing photothermal therapy on a target area in a subject comprising providing the nanovesicle described herein; administering the nanovesicle to the subject; and irradiating the nanovesicle at the target area with a wavelength of light, wherein the wavelength of light increases the temperature of nanovesicle.

According to a further aspect, there is provided a method of imaging a target area in a subject, comprising providing the nanovesicle described herein; administering the nanovesicle to the subject; irradiating the nanovesicle at the target area with a wavelength of light, wherein the nanovesicle emits a photoacoustic signal in response to the wavelength of light; and measuring and/or detecting the photoacoustic signal at the target area.

According to a further aspect, there is provided a method of imaging a target area in a subject, comprising providing the nanovesicle described herein; administering the nanovesicle to the subject; and measuring and/or detecting the fluorescence at the target area.

According to a further aspect, there is provided a method of performing photodynamic therapy at a target area in a subject, comprising providing the nanovesicle described herein; administering the nanovesicle to the subject; and allowing the porphyrin-lipid conjugate to disassociate from the nanovesicle at the target area; and irradiating the target area with a wavelength of light, wherein the wavelength of light activates the nanovesicle to generate singlet oxygen. In other embodiments, this method of performing photodynamic therapy further comprises irradiating the target area with a second wavelength of light, different from the first wavelength of light, to perform photothermal therapy. Preferably, the light is a continuous beam. In other embodiments, this method of performing photodynamic therapy further comprises, following the step of administering the nanovesicle to the subject, monitoring the delivery of the nanovesicle according to the method described herein.

According to a further aspect, there is provided a method comprising a combination of any of the methods described herein.

According to a further aspect, there is provided the methods described herein, wherein the nanovesicle further comprises a targeting molecule as described herein, and the targeting molecule targets the target area.

According to a further aspect, there is provided the methods described herein, further comprising allowing the porphyrin-lipid conjugate to accumulate at the target area.

Preferably, the target area is, but not limited to, a tumour.

According to a further aspect, there is provided a use of the nanovesicle described herein for performing photodynamic therapy.

According to a further aspect, there is provided a use of the nanovesicle described herein for performing photothermal therapy.

According to a further aspect, there is provided a use of the nanovesicle described herein for performing photoacoustic imaging.

According to a further aspect, there is provided a use of the nanovesicle described herein for performing fluorescence imaging.

The following examples are illustrative of various aspects of the invention, and do not limit the broad aspects of the invention as disclosed herein.

### EXAMPLES

Methods

Materials

Dipalmitoylphosphatidylcholine (DPPC), 1,2-dipalmitoyl-sn-glycero-3-phosphoethanolamine-N-[methoxy(polyethylene glycol)-2000] (DPPE-mPEG2000), 1-hexadecanoyl-sn-glycero-3-phosphocholine (PHPCPHPC) were purchased from Avanti Polar Lipids, Inc. (Alabaster, Ala.) and other chemicals and solvents were purchased from Sigma Aldrich. Flash column chromatography was performed with silica gel 60 (230-400 um) (Merck) or with diol modified silica (Sorbtech). Proton nuclear magnetic resonance spectra were collected on a Bruker Ultra Shield 400 PlusShield 400 Plus (400 MHz). HPLC and mass spectrometry was conducted on Waters Micro Mass HPLC. Extruder drain discs and polycarbonate membranes were purchased from Whatman (Piscataway, N.J.).

### Synthesis—Scheme 1

Compounds were synthesized according to Scheme 1 below. All synthesis procedures were conducted under dim light conditions.

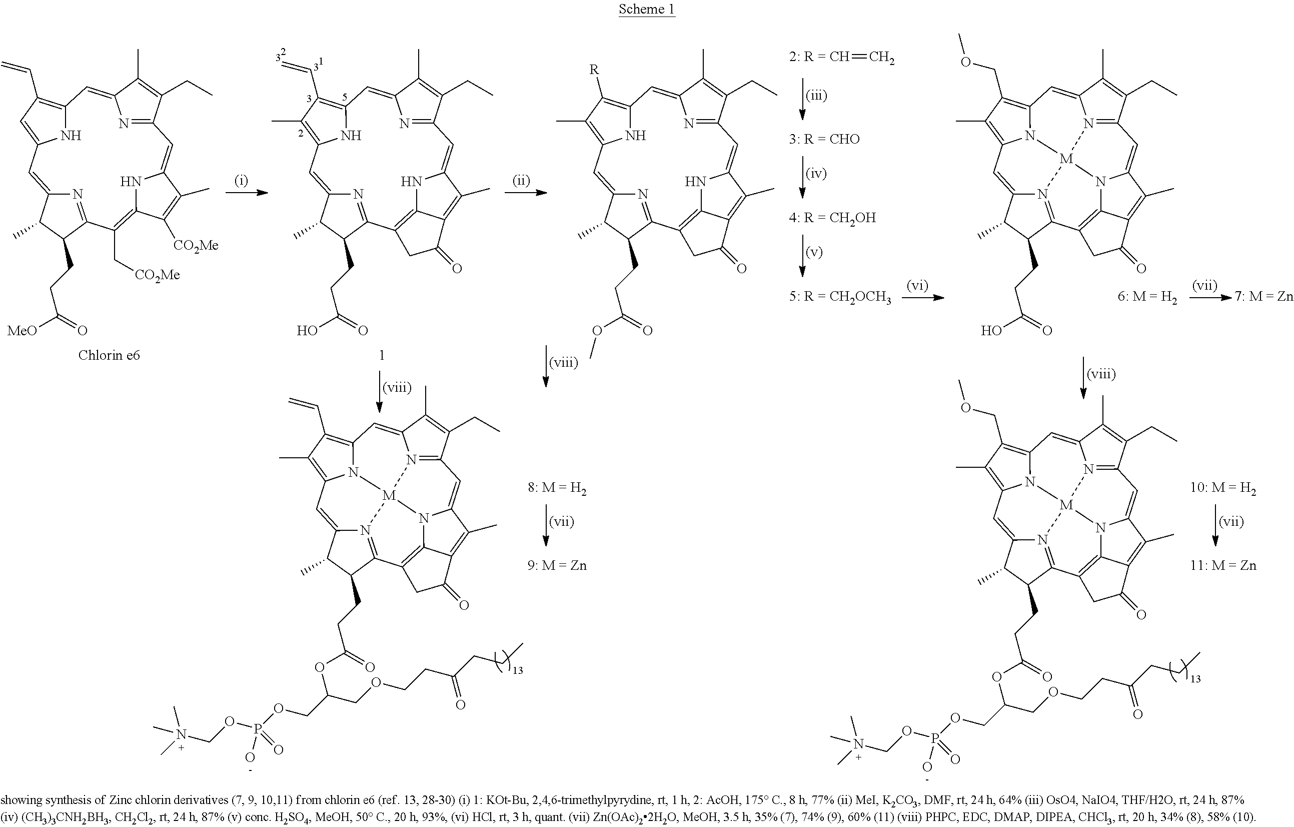

### Synthesis of Pyropheophorbide-a (1)

Chlorin e₆ trimethyl ester (176.9 mg, 0.28 mmol) was dissolved in dry 2,4,6-trimethylpyridine (15 mL), carefully degassed with Ar at 50° C. under vacuum and then cooled down to the room temperature. Potassium tert-butoxide (1 M in tert-butyl alcohol, 2.5 mL, 2.5 mmol) was added. After stirring at room temperature for 1 h, the reaction mixture was quenched with degassed glacial acetic acid (5 mL) at 0° C. Acetic acid along with a small amount of 2,4,6-trimethylpyridine was removed by distilled at 175° C. 2,4,6-trimethylpyridine (10 mL) was added again and the reaction mixture was refluxed at 175° C. under Ar for 8 h. The solvent was again removed as above. The residue was dissolved in chloroform, extracted five times with water, dried over Na₂SO₄ and filtered. Solvent was evaporated and crude product was dissolved in minimal amount of dichloromethane and hexane (125 ml) was slowly added. The product was recrystallized for 3 days at 4° C. and filtered.

Purple powder (114.7 mg, 0.22 mmol, 77%); ¹H NMR (CDCl₃, 400 MHz) δ=9.36 (1H, s, H5), 9.26 (1H, s, H10), 8.52 (1H, s, H20), 7.93 (1H, dd, J=17.7, 11.7 Hz, H3¹), 6.24 (1H, d, J=17.8 Hz, trans-3²CH═CH₂), 6.13 (1H, d, J=11.5 Hz, cis-3²CH═CH₂), 5.20 (2H, ABX, H13²), 4.47 (1H, m, H18), 4.28 (1H, m, H17), 3.60 (2H, m, H8¹), 3.59, 3.37, and 3.16 (3H, each s, H12¹, H2¹ and H7¹), 2.79-2.59 and 2.37-2.31 (4H, 2m, 17¹-CH₂CH₂), 1.81 (3H, d, J=7.3 Hz, H18¹), 1.65 (3H, t, J=7.7 Hz, H8²), 1.28 (1H, s, NH), −1.76 (1H, s, NH); ESI+MS m/z calculated for C₃₃H₃₅N₄O₃ [M+H]⁺ 535, found 535.

### Synthesis of Methyl 13²-demethoxycarbonylpheophorbide-a (2; methyl pyro-pheophorbide-a)

Potassium carbonate (271 mg, 2.0 mmol) and methyl iodide (60 uL, 0.95 mmol) were added to pyropheophorbide-a (1) (340 mg, 0.63 mmol) in DMF (50 mL) at 0° C., After 10 min the mixture was stirred at room temperature for 24 h and quenched with water and extracted with DCM. The combined organic layers were washed with brine, dried over Na₂SO₄, filtered and concentrated under reduced pressure. The crude product was purified by silica gel chromatography (DCM/Acetone=99/1).

Purple powder (223.7 mg, 0.41 mmol, 64%); ¹H NMR (400 MHz, CDCl₃) δ=9.44 (1H, s, H5), 9.33 (1H, s, H10), 8.55 (1H, s, H20), 7.98 (1H, dd, J=17.8, 11.5 Hz, H3′), 6.28 (1H, d, J=18.1 Hz, trans-3²CH═CH₂), 6.16 (1H, d, J=12.3 Hz, cis-3²CH═CH₂), 5.19 (2H, ABX, H13²), 4.49 (1H, m, H18), 4.30 (1H, m, H17), 3.65, 3.63, 3.41, 3.21 (12H, each s, H2¹, H12¹, 17³-CO₂CH₃, H7¹), 3.60 (2H, m, H8¹), 2.68-2.74 and 2.24-2.36 (4H, 2m, 17′-CH₂CH₂), 1.83 (3H, d, J=7.3 Hz, H18¹), 1.68 (3H, t, J=7.7 Hz, H8²), 0.41 (1H, s, NH), −1.73 (1H, s, NH); ESI+MS m/z calcd for C₃₃H₃₇N₄O₄ [M+H]⁺ 549, found 549.

### Synthesis of Methyl 3-devinyl-3-formyl-13²-demethoxycarbonylpheophorbide-a (3; methyl pyropheophorbide-d)

To methyl pyropheophorbide-a (2) (280 mg, 0.51 mmol) in THF (80 mL) at 0° C. was added 4 weight % osmium tetradoxide (108 ul, 108 umol) and then slowly a solution of sodium periodate (660 mg, 3.1 mmol) in water (30 mL). After 10 min the mixture was stirred at room temperature for 24 h under Ar, quenched with saturated sodium thiosulfate (80 mL) and extracted with DCM. The combined organic layers were washed with water and brine, dried over Na₂SO₄, filtered and concentrated under reduced pressure. The crude product was purified by silica gel chromatography (gradient; DCM/Acetone=99/1 to 95/5).

Orange powder (244.7 mg, 0.44 mmol, 87%); ¹H NMR (400 MHz, CDCl₃) δ=11.37 (1H, s, CHO), 9.95 (1H, s, H5), 9.30 (1H, s, H10), 8.76 (1H, s, H20) 5.25 (2H, ABX, H13²), 4.56 (1H, m, H18), 4.36 (1H, m, H17), 3.69 (2H, s, H8′), 3.66, 3.58, 3.50, 3.10 (12H, each s, H2¹, H12¹, 17³-CO₂CH₃, H7′), 2.70-2.56 and 2.22-2.41 (4H, 2m, 17¹-CH₂CH₂), 1.87 (1H, d, J=7.2 Hz, H18¹), 1.60 (3H, t, J=7.6 Hz, H8²), −0.47 (1H, s, NH), −2.39 (1H, s, NH); ESI+MS m/z calcd for C₃₃H₃₅N₄O₄ [M+H]⁺ 551, found 551.

### Synthesis of Methyl 3-devinyl-3-hydroxymethyl-13²-demethoxycarbonylpheophorbide-a (4)

To the aldehyde of 3 (244 mg, 0.44 mmol) in CH₂Cl₂ (80 mL) at 0° C. was added borane tert-butyl amine complex (38.8 mg, 0.44 mmol). After 10 min the mixture was stirred at room temperature for 24 h under Ar, quenched with 5% aqueous HCl at 0° C. and washed with 5% aqueous HCl, water, sat. NaHCO₃, and brine in succession. The extract was dried over Na₂SO₄, filtered and concentrated under reduced pressure. The crude product was purified by silica gel chromatography (gradient; DCM/Acetone=98/2 to 95/5).

Purple powder (211.9 mg, 0.38 mmol, 87%); 1H NMR (400 MHz, CDCl₃) δ 9.39 (1H, s, H5), 9.36 (1H, s, H10), 8.53 (1H, s, H20), 5.86 (1H, s, H3¹), 5.08 (2H, ABX, H13²), 4.44 (1H, m, H18), 4.20 (1H, m, H17), 3.66 (2H, s, H8′), 3.64, 3.57, 3.40, 3.24 (12H, each s, H2¹, H12¹, 17³-CO₂CH₃, H71), 2.64-2.53 and 2.31-2.18 (4H, 2m, 17′-CH₂CH₂), 1.77 (1H, d, J=7.2 Hz, CH-18¹), 1.67 (3H, t, J=7.6 Hz, CH₃-8²), 0.11 (1H, s, NH), −1.91 (1H, s, NH); ESI+MS m/z calcd for C₃₃H₃₇N₄O₄ [M+H]⁺ 553, found 553.

### Synthesis of Methyl 3-Devinyl-3¹-methoxymethyl-13²-demethoxycarbonylpheophorbide-a (5)

To chlorin (4) (23.8 mg, 0.043 mmol) in anhydrous MeOH (27 ml) was added conc. H₂SO₄ (2 ml). Upon addition, the color of the reaction mixture immediately turned a blue color. After refluxing at 50° C. under Ar for 20 h, the reaction mixture was cooled to room temperature and extracted with DCM and sat. NaHCO₃ until the solution turned purple. The organic layers were combined, washed with brine, dried over Na₂SO₄, filtered and concentrated under reduced pressure. The crude product was purified by silica gel chromatography (DCM/Acetone=98/2).

Purple powder (211.9 mg, 0.38 mmol, 87%); 1H NMR (400 MHz, CDCl₃) δ 9.53 (1H, s, H5), 9.43 (1H, s, H10), 8.57 (1H, s, H20), 5.69 (1H, s, H3′), 5.18 (2H, ABX, H13²), 4.50 (1H, m, H18), 4.31 (1H, m, H17), 3.70 (2H, s, H8¹), 3.68, 3.67, 3.63, 3.41, 3.26 (15H, each s, H2¹, H12¹, 3′-OCH₃, 17³-CO₂CH₃, H7¹), 2.75-2.68 and 2.62-2.54 (4H, 2m, 17¹-CH₂CH₂), 1.83 (1H, d, J=7.2 Hz, CH-18¹), 1.70 (3H, t, J=7.6 Hz, CH₃-8²), 1.27 (1H, s, NH), −1.73 (1H, s, NH); ESI+MS m/z calcd for C₃₄H₃₉N₄O₄ [M+H]⁺ 567, found 567.

### Synthesis of 3-Devinyl-3¹-methoxymethyl-13²-demethoxycarbonylpheophorbide-a (6)

To chlorin (5) (17.2 mg, 0.030 mmol) was added conc. HCl (2 ml) at 0° C. After 10 min, the mixture was stirred at room temperature for 3 h and extracted with DCM and sat. NaHCO₃. The organic layers were combined was dried over Na₂SO₄, filtered and concentrated under reduced pressure. The crude product was purified by diol silica gel chromatography (DCM/MeOH=90/10).

Purple powder (17.0 mg, 0.030 mmol, quant.); 1H NMR (400 MHz, CDCl₃) d 9.46 (1H, s, H5), 9.39 (1H, s, H10), 8.54 (1H, s, H20), 5.65 (1H, s, H3¹), 5.18 (2H, ABX, H13²), 4.48 (1H, m, H18), 4.30 (1H, m, H17), 3.68 (2H, s, H8¹), 3.67, 3.63, 3.39, 3.24 (12H, each s, H2¹, H12¹, 3¹-OCH₃, H71), 2.70-2.61 and 2.38-2.36 (4H, 2m, 17¹-CH₂CH₂), 1.81 (1H, d, J=7.2 Hz, CH-18¹), 1.68 (3H, t, J=7.6 Hz, CH₃-8²), 0.87 (1H, s, NH), −1.72 (1H, s, NH); ESI+MS m/z calcd for C₃₃H₃₇N₄O₄ [M+H]J 553, found 553.

### Synthesis of Zinc 3-devinyl-3′-methoxymethy-13²-demethoxycarbonylpheophorbide-a (Zn-MeO-chlorin acid) (7)

To chlorin 6 (2.0 mg, 3.6 umol) in MeOH (3 mL), was added Zn(OAc)₂.2H₂O (11.3 mg, 44 umol) at 0° C. After 5 min, the mixture was stirred at room temperature for 3.5 h and extracted with DCM and H₂O. The combined organic layer was dried over Na₂SO₄, filtered, and concentrated under reduced pressure. The crude product was purified by diol silica gel chromatography (DCM/MeOH=90/10). The purity and identity was confirmed by HPLC and mass spectrometry (C₈ reverse phased column, 0.8 mL/min flow at 25° C. with acetonitrile/0.1% triethylammonium acetate=10/90 for initial two min and then gradually changed to 0/100 over 11 min followed by a 2 min hold). Compound eluted at 12.3 min.

Dark green powder (0.80 mg, 1.3 umol, 35%); UV/Vis (THF): λₘₐₓ(ε)=648 nm (93000 Lmol⁻¹ cm⁻¹); ESI+MS m/z calcd for C₃₃H₃₅N₄O₄Zn [M+H]⁺ 615, found 615.

### Synthesis of 3-Devinyl-3′-methoxymethyl-13²-demethoxycarbonylpheophorbide-a lipid (MeO-chlorin-lipid) (10)

To chlorin 6 (13.0 mg, 0.024 mmol) in anhydrous CHCl₃ (3 mL), was added DIPEA (2.04 μL, 0.012 mmol), DMAP (5.9 mg, 0.053 mmol), (16:0) PHPC (11.5 mg, 0.023 mmol), and EDC (8.7 mg, 0.045 mmol) successively. After stirring at room temperature under Ar for 20 h, the reaction mixture was extracted with DCM and sat. NH₄Cl and further extracted with water. The combined organic layer was dried over Na₂SO₄, filtered and concentrated under reduced pressure. The crude product was purified by diol silica gel chromatography (gradient; DCM/MeOH=99/1 to 90/10). The identity was confirmed by HPLC and mass spectrometry (C8 sunfire column, 0.8 mL/min flow at 60° C. with a solvent gradient, acetonitrile/0.1% trifluoroacetic acid=20/80 to 30/70 for initial two min and then gradually changed into 0/100 over another 14 min followed by a 5 min hold at the same ratio. Products eluted at 15.2 min and 15.4 min (acyl-migrated regioisomer products) together with remained PHPC which was eluted at 11.1 min.

Dark yellow powder (14.4 mg, 0.014 mmol, 58% calculated based on the absorbance in THF); UV/Vis (THF): λₘₐₓ(E)=661 nm (47000 Lmol⁻¹ cm⁻¹); ESI+MS m/z calcd for C₅₇H8₅N₄O₆P [M+H]⁺ 1031, found 1031.

### Synthesis of Zinc 3-Devinyl-3′-methoxymethyl-13²-demethoxycarbonylpheophorbide-a lipid (Zn-MeO-chlorin lipid) (11)

To lipid conjugated chlorin 10 (9.6 mg, 9.3 μmol) in MeOH (4 mL), was added Zn(OAc)₂.2H₂O (17.8 mg, 81 umol) at 0° C. After 5 min, the mixture was stirred at room temperature for 2 h and extracted with 1-butanol and H₂O. The combined organic layer was concentrated under reduced pressure. The identity was confirmed with HPLC and mass spectrometry (same protocol as for lipid conjugated chlorin 10 but chose 0.1% triethylammonium acetate as aqueous solvent instead of trifluoroacetic acid) Products eluted at 14.7 min and 15.0 min (acyl-migrated regioisomer products) together with remained PHPC which was eluted at 10.8 min.

Green powder (6.05 mg, 0.014 mmol, 60% calculated based on the absorption in THF); UV/Vis (THF): λₘₐₓ(ε)=648 nm (93000 Lmol⁻¹ cm⁻²); ESI+MS m/z calcd for C₅₇H₈₃N₄O₆P Zn [M+H]⁺ 1093, found 1093.

### Synthesis of 13²-demethoxycarbonylpheophorbide-a lipid (8)

To pyropheophorbide-a (1) (150 mg, 0.28 mmol) in anhydrous CHCl₃ (16 mL), was added DMAP (103 mg, 0.84 mmol), EDC (161 mg, 0.84 mmol), and PHPC (167 mg, 0.34 mmol). After stirring at room temperature under Ar for 19 h, the reaction mixture was concentrated under reduced pressure. Diol silica gel column chromatography was performed. (gradient; DCM/MeOH 100/0 to 97.2/2.8) Purity (>95%) and identity (acyl-migrated regioisomer product) was confirmed with HPLC and mass spectrometry (same protocol as for lipid conjugated chlorin 10.) Compound eluted at 17.8 min.

Dark yellow powder (96 mg, 0.094 mmol, 34%); UV/Vis (MeOH):λₘₐₓ(ε)⁼665 nm (45 000 L mol⁻¹ cm⁻¹); ESI+MS m/z calcd for C₅₇H₈₃N₄O₅P [M+H]⁺ 1013, found 1013.

### Synthesis of Zinc 13²-demethoxycarbonylpheophorbide-a lipid (Zn-vinyl-chlorin lipid) (9)

Pyropheophorbide a-lipid was synthesized as previously reported.²⁸ To pyropheophorbide a-lipid (6.2 mg, 6.2 μmol) in MeOH (3 mL), was added Zn(OAc)₂.2H₂O (15.3 mg, 70 μmol) at 0° C. After 5 min, the mixture was stirred at room temperature for 3.5 h and extracted with 1-BuOH and H₂O. The combined organic layer was dried over Na₂SO₄, filtered, and concentrated under reduced pressure. Purity and identity (acyl-migrated regioisomer product) were confirmed with HPLC and mass spectrometry (same protocol as lipid conjugated chlorin 10 but chose 0.1% triethylammonium acetate as aqueous solvent instead of trifluoroacetic acid) Compound eluted at 15.2 min.

Dark green powder (4.9 mg, 4.6 μmol, 74%); UV/Vis (MeOH):λₘₐₓ(E)=659 nm (78000 Lmol⁻¹ cm⁻¹); ESI+MS m/z calcd for C₅₇H₈₁N₄O₅PZn [M+H]⁺ 1076, found 1076.

Nanovesicle Formulation

Lipid film was prepared in the 12 mm×35 mm clear glass threaded vials (Fisher Scientific) by combining 20% of chlorin derivatives (either Zn-MeO-chlorin acid 7, Zn-vinyl-chlorin lipid, MeO-chlorin lipid, or Zn-MeO-chlorin lipid) with 75% DPPC and 5% DPPE-mPEG2000 dissolved in MeOH or chloroform. The lipid solutions were dried under a stream of nitrogen gas for 30 min and further dried under vacuum desiccation for at least 3 h before hydration. One milliliter phosphate buffered saline (PBS) was added to the lipid films and 10 freeze and thaw cycles was performed by sequentially freezing the sample in liquid nitrogen, followed by rapid thawing in a 70° C. water bath. Dispersed particles were then subjected to extrusion by passing samples 10 times through a high pressure extruder loaded with 100 nm polycarbonate filter membranes at 70° C.

Particle Characterization

Z-average size and polydispersity of the samples in PBS were measured using a Malvern ZS90 (Instruments, UK). Absorption spectra of each sample was determined by UV spectroscopy (CARY 50 UV/Vis S3 Spectrophotometer, Varian Inc.). Absorption spectra of intact samples was measured in PBS, and that of detergent disrupted samples was measured after adding Triton-X100 so that the final detergent concentration was 0.1% v/v.

The ratio of the amount of zinc chlorin derivatives incorporated into the particles to that of initially loaded was also determined was also determined as dye recovery % by the following equation:

$$\% dye incorporated into nanovesicles = ( \frac{{ABS}_{658 n m} ( after extrusion )}{{ABS}_{658 n m} ( before extrusion )} ) \times 100$$

where, ABS_{685 nm} (before extrusion) is the absorbance at 658 nm before extrusion and ABS_{685 nm} (after extrusion) is the absorbance at 658 nm after extrusion. These sample measurements were made by taking a small amount of sample before and after extrusion, each of which was dissolved by diluting 20 times in MeOH.

The color of Zn-MeO-chlorin acid (7) and Zn-MeO-chlorinchlorin lipid (11) nanoparticles was monitored after 10 freeze and thaw cycles. Concentration of chlorin dyes in each sample was adjusted to 60 μM. Samples were stored in the dark at room temperature for 10 days.

Fluorescence measurements were carried out Fluoromax ut on a Fluoromax-4 spectrofluorometer (Horiba Jobin Yvon, NJ) Fluorescence spectra of intact and detergent disrupted state was measured by exciting samples at 556 nm with a 5 nm slit width and collecting fluorescence from 600 nm to 800 with a 5 nm slit width. Quenching efficiency was calculated by the following equation:

$$\% quenching efficiency = ( 1 - \frac{{F}_{0}}{{F}_{detergent}} ) \times 100$$

Where F₀ and F_{detergent} are the integration of fluorescence intensity from 600 nm to 800 nm for intact and detergent-disrupted sample respectively. Circular dichroism spectra was measured in PBS and 0.1% Triton-X100 using a J-815 Circular Dichroism Spectrometer (JASCO Inc.).

Induction of Hamster Cheek Pouch Carcinoma Model

All procedures were approved by the animal care committee at the University Health Network. Six to eight week old male Syrian hamsters (Harlan, Indianapolis, USA) were used as a model of oral carcinoma in humans. To induce tumor growth, 0.5% 7,12-dimethylbenz(a)anthracene (DMBA) in DMSO was applied with a non-absorbent sponge to the inner mucosa of the right cheek while the animals were anesthetized with isofluorane. This procedure was repeated 3 times a week for a period of 16-20 weeks. Application of DMBA in this manner resulted in the generation of oral carcinoma between 5-10 mm in size after 16 weeks.

Photoacoustic Imaging of Hamster Cheek Pouch Carcinoma Model

Imaging of the chemically-induced hamster check pouch carcinoma was initiated by first anaesthetizing the animal with ketamine/xylazine through an intraperitoneal route. Once the animal reached an appropriate plane of anaesthesia, the check pouch was inverted to expose the tumor. The tumor was covered using ultrasound gel and the 21 MHz-centered photoacoustic transducer was placed over the tumor. Photoacoustic spectra (680-780 nm) of the tumor cross-section was acquired at 45 dB prior to injection and 5 min after a 115 nmol (dye content) intravenous administration of the (20%) Zn-MeO-chlorin-lipid liposome. An average photoacoustic spectra of the tumor was acquired by making a region-of-interest measurement of the tumor area (60 mm²) and exporting the spectra using the Vevo LAB software package (FujiFilm, Visualsonics, Toronto, ON). Pixel arithmetic was further carried out on the image cube by subtracting the photoacoustic intensity collected at 740 nm (endogenous contrast) from the nanoparticle photoacoustic signal peak at 725 nm (dye contrast) and applying a Gaussian smoothing filter (3 pixel by 3 pixel; σ=1) over the data to reduce noise.

Photoacoustic Signal Detection

For photoacoustic signal measurements, PBS, Zn-MeO-chlorin lipid (11) in PBS (for intact) or in 0.1% Triton-X100 were injected into polyethylene tubes (in. diam.: 0.381 mm; out. diam.: 1.09; PE20; Intramedic, BD), which was then placed in a plastic holder filled with water. Signals were obtained using a Vevo 2100 LAZR photoacoustic imaging system (Fujifilm, Toronto, ON) equipped with a 21 MHz-centered transducer and a flashlamp-pumped Q-switched Nd:YAG laser. Photoacoustic spectra was measured from 680 to 970 nm with a 1 nm step size. In order to measure concentration dependency of photoacoustic signal of Zn-MeO-chlorin lipid (11), 5 different concentration was prepared. Optical density at Q_{y} maxima of each sample in MeOH (653 nm) was adjusted to 0, 1.7, 3.3, 5.0, and 6.6. For this measurement, measurement, laser was set to 725 nm. Images were generated by scanning across the length of the tube and collecting the signal originating from peak at 725 nm.

### Synthesis—Scheme 2

Compounds were synthesized according to Scheme 2 below. All synthesis procedures were conducted under dim light conditions. Detailed synthesis of compounds 2, 5, 6, 11, 14 and 15 are described above (structures renumbered in Scheme 2). Synthesis of compounds 12-13 are underway. Synthesis of compounds 3, 4, 7-10, 16 and 17 are detailed below.

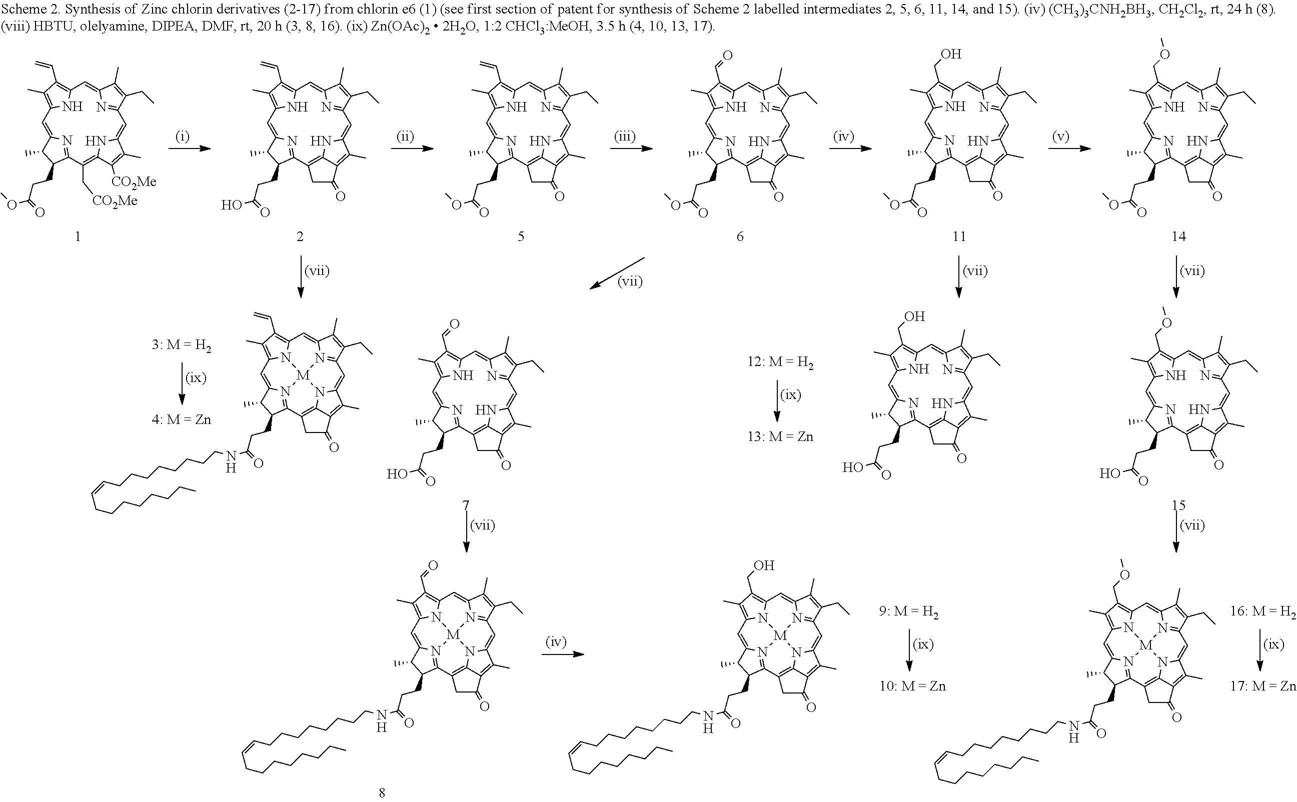

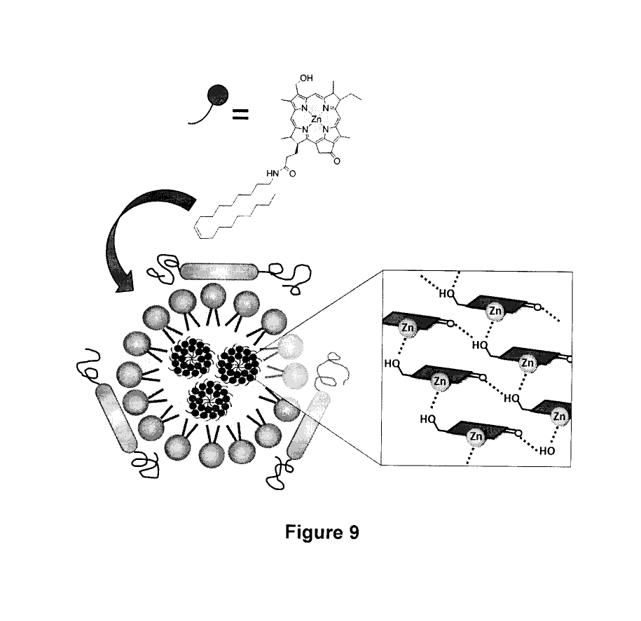

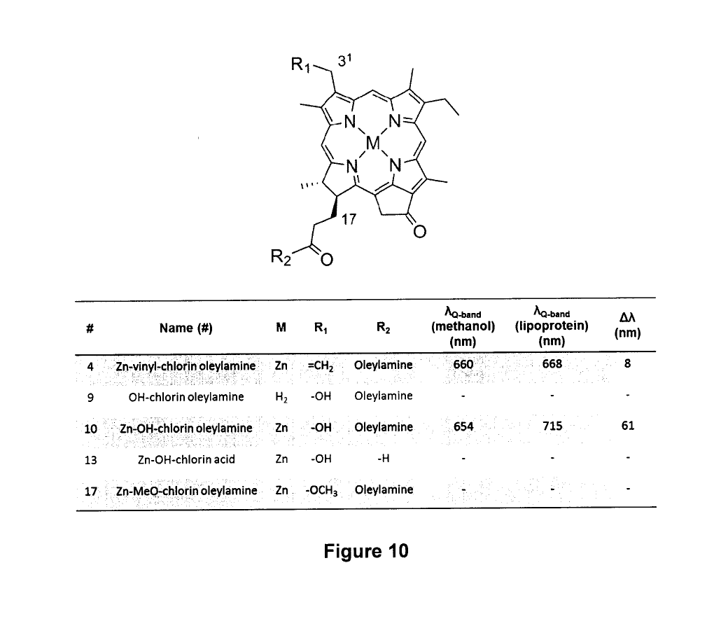

An example of zinc chlorin oleylamine molecules assembled within the core of a lipoprotein nanoparticle is illustrated schematically in FIG. 9. A summary of the generalized structure for the series of chlorin molecules studied/to-be studied in lipoprotein nanoparticles are found in FIG. 10.

### Synthesis of 13²-demethoxycarbonylpheophorbide-a oleylamine (Vinyl-chlorin oleylamine) (3)

Pyropheophorbide-a (2) (30.0 mg, 0.056 mmol) in anhydrous DMF (800 μL) was added to HBTU activating agent (25.5 mg, 0.118 mmol, 1.2 mol eq.), olelyamine (19.0 mg, 23.5 μL, 0.071 mmol, 1.2 mol eq.) and DIPEA (20 μL) for basic conditions. After stirring at room temperature overnight in a closed vial, the reaction mixture was extracted with 2:1 CHCl₃:MeOH and washed with DI H₂O five times. The combined organic layer was dried over Na₂SO₄, filtered and concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 4.406 min.

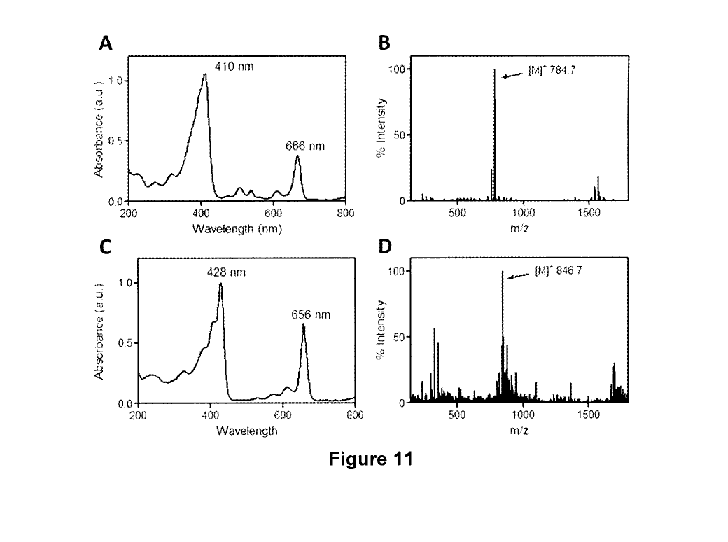

ESI+MS m/z calcd for C₅₁H₆₉N₅O₂ [M+H]⁺ 786, found 785. See FIG. 11.

### Synthesis of Zinc 13²-demethoxycarbonylpheophorbide-a oleylamine (Zn-vinyl-chlorin oleylamine) (4)

To oleylamine-conjugated chlorin 3 (23.5 mg, 30 μmol) in 1:2 CHCl₃:MeOH (9 mL total), was added Zn(OAc)₂.2H₂O (91.2 mg, 0.45 mmol, 15 mol eq.) at 0° C. After 5 min, the mixture was stirred at room temperature for 3.5 h and extracted with 2:1 CHCl₃:MeOH and DI H₂O five times. The combined organic layer was concentrated under reduced pressure The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 3.991 min.

ESI+MS m/z calcd for C₅₁H₆₇N₅O₂Zn [M+H]⁺ 846, found 847. See FIG. 11.

### Synthesis of 3-devinyl-3-hydroxymethyl-13-demethyl-13²-demethoxycarbonylpheophorbide-a (Aldehyde chlorin acid) (7)

To chlorin 6 was added conc. HCl (2 ml) at 0° C. After 10 min, the mixture was stirred at room temperature for 3 h and extracted with DCM and sat. NaHCO₃. The organic layers were combined was dried over Na₂SO₄, filtered and concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 2.502 min

ESI+MS m/z calcd for C₃₂H₃₂N₄O₄ [M+H]⁺ 536, found 537.

### Synthesis of Methyl 3-devinyl-3-formyl-13²-demethoxvcarbonvlpheophorbide-a oleylamine (Aldehyde-chlorin oleylamine) (8)

Chlorin 7 in anhydrous DMF (800 μL) was added to HBTU activating agent (2.5 mol equivalents), DIPEA (<3% v/v) and olelyamine (1 mol equivalent). After stirring at room temperature overnight in a closed vial, the reaction mixture was extracted with 2:1 CHCl₃:MeOH and DI H₂O five times. The combined organic layer was dried over Na₂SO₄, filtered and concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 4.130 min.

ESI+MS m/z calculated for C₅₀H₆₇N₅O₃ [M+H]⁺ 786, found 787.

### Synthesis of 3-Devinyl-3′-hydroxyl-13²-demethoxycarbonylpheophorbide-a oleylamine (OH-chlorin oleylamine) (9)

To the aldehyde of 8 in CH₂Cl₂ (80 mL) at 0° C. was added borane tert-butyl amine complex (1 mol equivalent). After 10 min the mixture was stirred at room temperature for 24 h under Ar, quenched with 5% aqueous HCl at 0° C. and washed with 5% aqueous HCl, water, sat. NaHCO₃, and brine in succession. The extract was dried over Na₂SO₄, filtered and concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 3.724 min.

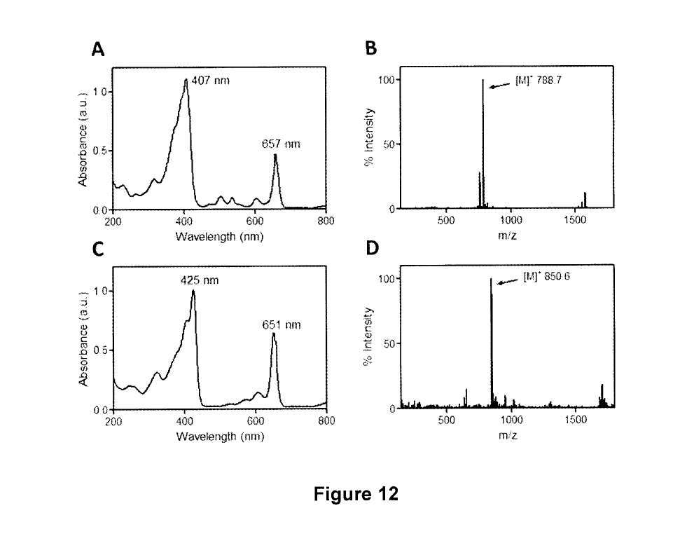

ESI+MS m/z calculated for C₅₀H₆₉N₅O₃ [M+H]⁺ 788, found 789. See FIG. 12.

### Synthesis of Zinc 3-Devinyl-3¹-hydroxyl-13²-demethoxycarbonylpheophorbide-a oleylamine (Zn—OH-chlorin oleylamine) (10)

To oleylamine-conjugated chlorin 9 in 1:2 CHCl₃:MeOH, was added Zn(OAc)₂.2H₂O (15 mol eq.) at 0° C. After 5 min, the mixture was stirred at room temperature for 3.5 h and extracted with 2:1 CHCl₃:MeOH and DI H₂O five times. The combined organic layer was concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 3.422 min.

ESI+MS m/z calculated for C₅₀H₆₇N₅O₃Zn [M+H]⁺ 850, found 851. See FIG. 12.

### Synthesis of 3-Devinyl-3¹-methoxymethyl-13²-demethoxycarbonylpheophorbide-a oleylamine (MeO-chlorin-oleylamine) (16)

Chlorin 15 (32.8 mg, 0.059 mmol) in anhydrous DMF (800 μL) was added to HBTU activating agent (47.2 mg, 0.125 mmol), DIPEA (20 μL) and olelyamine (19.0 mg, 23.5 μL, 0.071 mmol). After stirring at room temperature overnight in a closed vial, the reaction mixture was extracted with 2:1 CHCl₃:MeOH and DI H₂O five times. The combined organic layer was dried over Na₂SO₄, filtered and concentrated under reduced pressure. The crude product was purified by diol silica gel chromatography (gradient; DCM/MeOH=97/3). The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 4.015 min.

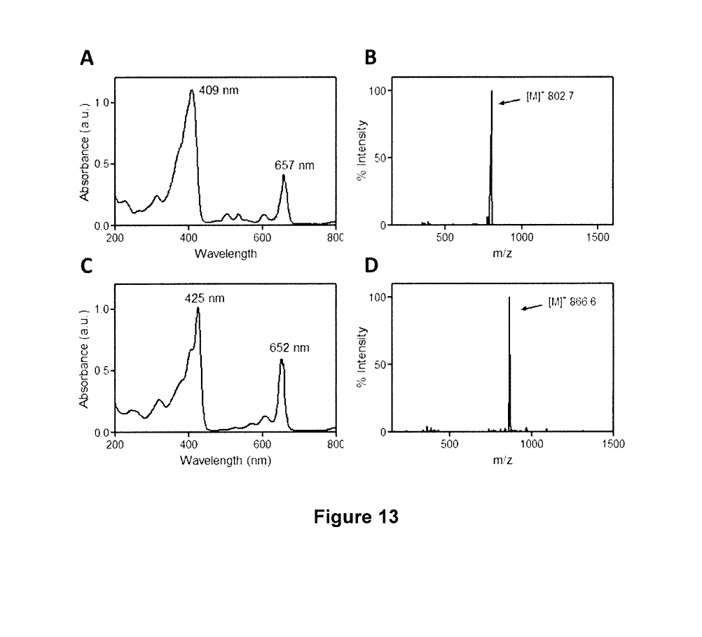

ESI+MS m/z calculated for C₅₁H₇₁N₅O₃ [M+H]⁺ 802, found 803. See FIG. 13.

### Synthesis of Zinc 3-Devinyl-3-methoxymethyl-13²-demethoxvcarbonvlpheophorbide-a oleate (Zn-MeO-chlorin oleylamine) (17)

To oleylamine-conjugated chlorin 16 (21.7 mg, 27.1 μmol) in 1:2 CHCl₃:MeOH (9 mL total), was added Zn(OAc)₂.2H₂O (89.0 mg, 0.41 mmol, 15 mol eq.) at 0° C. After 5 min, the mixture was stirred at room temperature for 3.5 h and extracted with 2:1 CHCl₃:MeOH and DI H₂O five times. The combined organic layer was concentrated under reduced pressure. The compound identity was confirmed by UPLC and mass spectrometry (C8 sunfire column, 0.6 mL/min flow at 60° C. with a gradual solvent gradient, 0.1% formic acid in water/acetonitrile=60/40 to 0/100 for three min with a hold at the same ratio for another 1 min and then gradually changed back to 60/40 over the last 1 min. Product eluted at 3.780 min.

ESI+MS m/z calculated for C₅₁H₆₉N₅O₃Zn [M+H]⁺ 864, found 867. See FIG. 13.

High Density Lipoprotein Nanoparticle Formulation

Lipid film was prepared in 12 mm×75 mm clear glass threaded vials (Fisher Scientific) by combining 0.1 μmol (4, 10, 10, 13 or 17) and 1.0 μmol DMPC in chloroform. The lipid solutions were dried under a slow stream of nitrogen gas for 30 min and further dried under a higher stream for at least 1 h before hydration. One mL of phosphate buffered saline (PBS) was added to the dried lipid films and sonicated in a 47° C. water bath for 10 minutes immediately followed by Bioruptor at low frequency (30 kHz) for 30 cycles (30 s on/30 s off) at 40° C. R4F peptide (0.1 μmol; 1 mg/mL in PBS) was added into the rehydrated solution and the mixture was mixed gently at 4° C. overnight. The solution was centrifuged at 13 500 rpm for 20 min subsequently and filtered with a 0.1 μm membrane. Unbound peptide was removed and nanoparticle sample was concentrated using a 10 kDa membrane centrifugal filter (EMD Millipore) by two rounds of centrifugation at 4000 g for 7 min at 4° C.

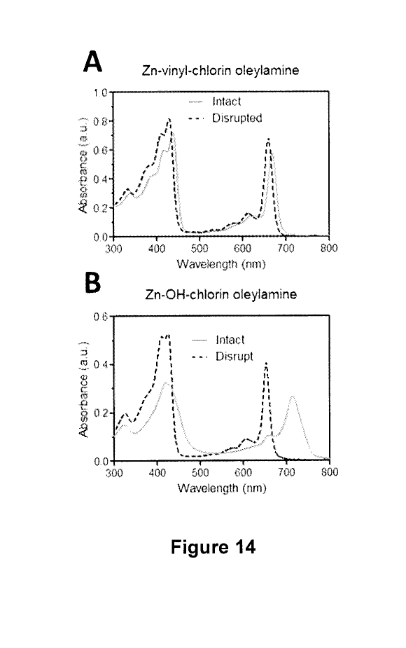

The UV-Vis absorption spectra of lipoprotein nanoparticles with Zn-containing oleylamine compounds 4, 10 and 17 are shown in FIG. 14. The intact structures are labeled in green (solid line) and methanol-disrupted structures are labeled in black (dashed line).

Photoacoustic Signal Detection

For photoacoustic signal measurements, PBS, Zn—OH-chlorin oleylamine (10) in PBS (for intact) or in 10% Triton-X100 were injected into polyethylene tubes (in. diam.: 0.381 mm; out. diam.: 1.09; PE20; Intramedic, BD), which was then placed in a plastic holder filled with water. Signals were obtained using a Vevo 2100 LAZR photoacoustic imaging system (Fujifilm, Toronto, ON) equipped with a 21 MHz-centered transducer and a flashlamp-pumped Q-switched Nd:YAG laser. Photoacoustic spectra was measured from 680 to 850 nm with a 1 nm step size. In order to measure concentration dependency of photoacoustic signal of Zn—OH-chlorin oleylamine (10), 5 different concentrations was prepared (0, 10, 20, 40 and 80 μM). For this measurement, the laser was set to 715 nm.

Results and Discussion

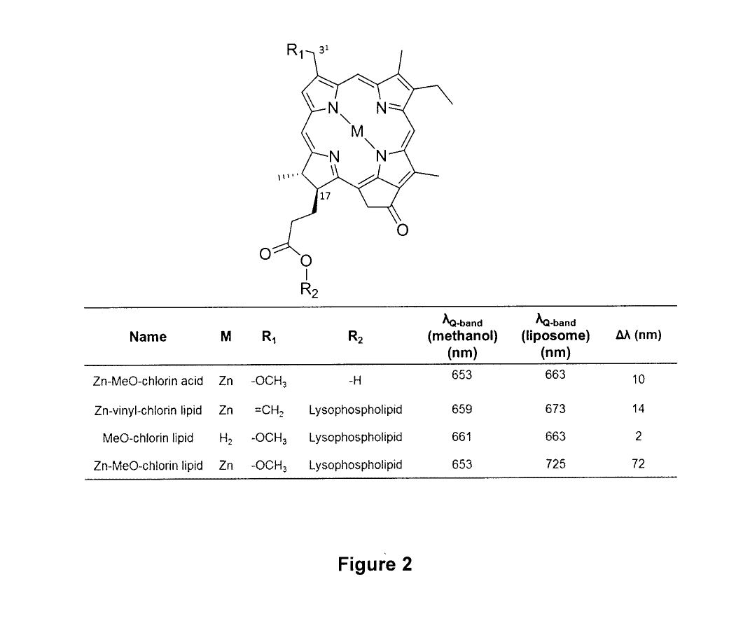

Based earlier published studies^{1, 12,16, 21, 22}, it is hypothesized that π-π interaction and axial metal coordination between chlorin molecules would be important to form ordered chlorin aggregation in the lipid membrane like those observed in chlorosomes. Three modifications on the pyropheophorbide a chlorin structure were made including: (i) a methoxy group at the 3¹ position, (ii) a centrally-coordinated zinc atom, and (iii) conjugation of a lysophospholipid to a chlorin molecule at the 17-position, were synthesized and investigated their effect on the formation of self-assembled chlorin aggregates within a liposome environment (FIG. 2). Firstly, 3¹-vinyl-13¹-oxo-chlorin acid 1 and 3¹-methoxy-13′-oxo-chlorin acid 7 were synthesized from chlorin e₆ (Scheme 1). These products were subsequently conjugated with 1-palmitoyl-2-hydroxy-sn-glycero-3-phosphocholine (PHPC) to get 3¹-vinyl-13′-oxo-chlorin lipid 8 and 3¹-methoxy-13′-oxo-chlorin lipid 10, respectively. Zinc insertion into chlorin acid 6 resulted in zinc 3′-methoxy-13′-oxo-chlorin acid (Zn-MeO-chlorin acid) 7, while insertion into 3¹-vinyl-13′-oxo-chlorin lipid 8 and 3¹-methoxy-13′-oxo-chlorin lipid (MeO-chlorin lipid) 10 gave zinc 3′-vinyl-13′-oxo-chlorin lipid (Zn-vinyl-chlorin lipid) 9 and zinc 3¹-methoxy-13′-oxo-chlorin lipid (Zn-MeO-chlorin lipid) 11, respectively.

For comparison of structure dependent aggregation of chlorin derivatives in lipid membranes, we studied four chlorin analogs: Zn-MeO-chlorin acid, Zn-vinyl-chlorin lipid, MeO-chlorin lipid, and Zn-MeO-chlorin lipid (FIG. 2). These dyes were combined with dipalmitoylphosphatidylcholine (DPPC) and PEGylated phospholipid and formulated using the freeze-thaw extrusion procedure to prepare lipid nanovesicles²³. Dynamic light scattering (DLS) showed that the hydrodynamic diameter of all the chlorin derivatives embedded in liposomal membranes was approximately 100 nm. (See Table S1 below)

TABLE S1
Size measurements of 20% chlorin derivatives embedded
within liposomes using dynamic light scattering. (A)
Z-average measurements and (B) Polydispersity index.

| | Z-average | |
|---|---|---|
| Name | (d · nm) | PDI |
| Zu—MeO-chlorin acid | 113.9 ± 9.6 | 0.3 ± 0.2 |
| Zn-vinyl-chlorin lipid | 101 ± 2.3 | 0.09 ± 0.05 |
| MeO-chlorin lipid | 105 ± 1.6 | 0.09 ± 0.08 |
| Zn—MeO-chlorin lipid | 96.0 ± 3.9 | 0.10 ± 0.04 |

The polydispersity index (PDI), which provides a measure of the homogeneity of the extruded nanovesicles showed that samples prepared with Zn-vinyl-chlorin lipid, MeO-chlorin lipid and Zn-MeO-chlorin lipid possessed a PDI less than 0.1, suggesting that the nanovesicles were homogeneously dispersed. However, nanovesicles using the Zn-MeO-chlorin acid gave a much larger PDI (>0.3), indicating the existence of several particle sizes in the sample.

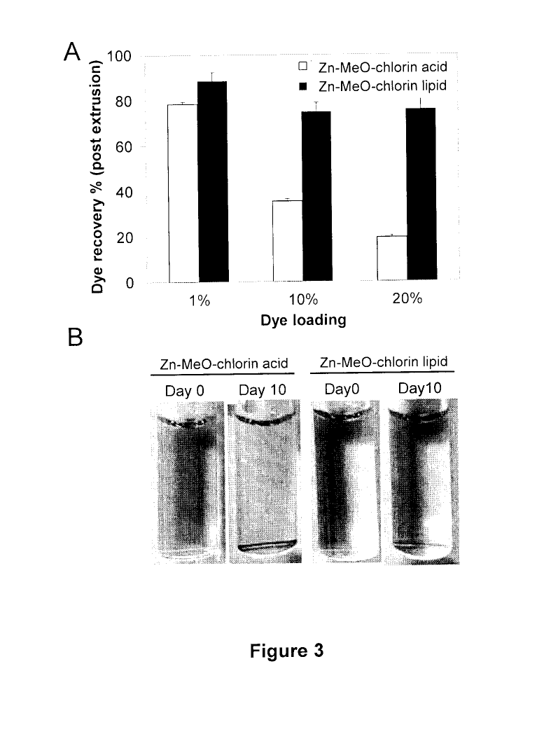

In addition to hydrodynamic size, we also compared the stability of the nanovesicles prepared using the Zn-MeO-chlorin acid versus the comparable lipid conjugate. In order to compare differential stability, we examined the dye recovery from both samples extruded to form lipid nanovesicles. Zn-MeO-chlorin acid displayed a lower recovery than Zn-MeO-chlorin lipid at loading percentages varying from 1 to 20 mol % (FIG. 3, A). At the highest loading percentage, Zn-MeO-chlorin acid only displayed a 20% recovery rate, while all lipid conjugated chlorin derivatives had 70% recovery rate (FIG. 3, A & FIG. 14). We also monitored the stability of Zn-MeO-chlorin lipid and Zn-MeO-chlorin acid samples stored at room temperature after freeze-thaw assisted rehydration (FIG. 3, B). Both samples appeared homogeneously dispersed in PBS immediately after the freeze-thaw procedure. However, Zn-MeO-chlorin acid flocculated after 10 days in contrast to the Zn-MeO-chlorin lipid which remained visibly dispersed over the same time period. These results indicate that lipid conjugation improves the stability of the dye within lipid membranes, possibly by facilitating intercalation within the lipid bilayers as opposed to the formation of large insoluble random aggregates between chlorin dyes.

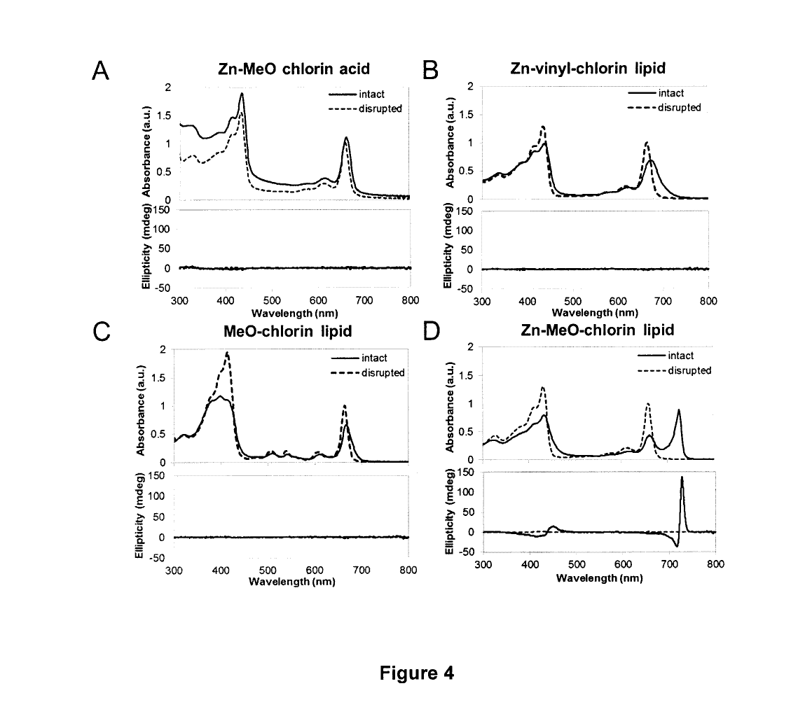

The optical properties of nanovesicles loaded with 20% Zn-vinyl-chlorin lipid, MeO-chlorin lipid, Zn-MeO-chlorin lipid or 1% Zn-MeO-chlorin acid were investigated by UV/visible spectrophotometry and circular dichroism (CD). We chose 1% loading for the Zn-MeO-chlorin acid sample because recovery after extrusion was found to be low when the dye was loaded at 10% and 20%. This was likely caused by insolubility of the dye, which induces formation of large insoluble aggregates which are unable to pass the filter membrane. Zn-MeO-chlorin acid did not demonstrate appreciable shift in optical absorption nor CD at 1% loading (FIG. 4, A). Chlorin-lipid samples that possessed only one modification, 3¹-methoxy substitution (FIG. 4, B) or the Zn coordination (FIG. 4, C), only showed a slight red-shift and a broadening of the absorption spectrum. These samples also did not display an appreciable absorption of circularly polarized light. In contrast, the absorption spectra of Zn-MeO-chlorin lipid showed a 72 nm bathochromic shift in the lowest energy absorption band (Q_{y}) compared to the absorption in its monomeric state in methanol or in 0.1% Triton X-100 (FIG. 4, D). Its CD spectra also showed a bisignate spectra around the Q_{y} band region while the signal was eliminated when the nanovesicles were treated with detergent (FIG. 4, D). This curved spectroscopic structure indicates the presence of exciton coupling between chirally assembled Zn-MeO-chlorin lipid molecules.^{19, 24} In addition to the far red-shifted peak at 725 nm, a secondary peak could be observed that was only 3 nm red-shifted from the monomer absorption. This peak could be caused by the presence of monomeric dyes that do not participate in ordered aggregation. Indeed, this absorption band did not display circular polarized light absorption, which would be indicative of chiral ordered dye assemblies. Additionally, Zn-MeO-chlorin lipid in lipid nanovesicles showed no appreciable Stokes' shift (see Table S2 below).

TABLE S2
Fluorescence properties of zinc chlorin derivatives
embedded within lipid nanovesicles.

| | | | Full width |
|---|---|---|---|
| | FL λₘₐₓ | Stokes Shift | at half max |
| Name | (nm) | (nm) | (nm) |
| Zn—MeO-chlorin acid | 664 | 1 | 24 |
| Zn-vinyl-chlorin lipid | 682 | 9 | 21 |
| MeO-chlorin lipid | 676 | 10 | 40 |
| Zn—MeO-chlorin lipid | 725 | 0 | 15 |

This phenomenon, has been reported in both naturally isolated or synthetically assembled ordered aggregates.²⁵ Zn-MeO-chlorin acid also displayed a small Stokes shift (1 nm), while Zn-vinyl-chlorin lipid and MeO-chlorin lipid displayed a more substantial Stokes shift of 9 and 10 nm, respectively (see Table S2). Of the entire series tested, only Zn-MeO-chlorin acid and Zn-MeO-chlorin lipid possessed both a centrally coordinated zinc metal and 3¹-oxygen, which allows them to form a Zn . . . 31-coordination bond. In comparison, Zn-vinyl-chlorin lipid and MeO-chlorin lipid lack either a 3¹-oxygen or central metal (FIG. 2). Considering the result that nanovesicles made with Zn-MeO-chlorin lipid showed ordered aggregation while Zn-vinyl-chlorin lipid and MeO-chlorin lipid did not, metal coordination involving the chelated Zn and a 3¹-methoxy substituent is a key interaction that governs the formation of ordered aggregates in the lipid bilayer.

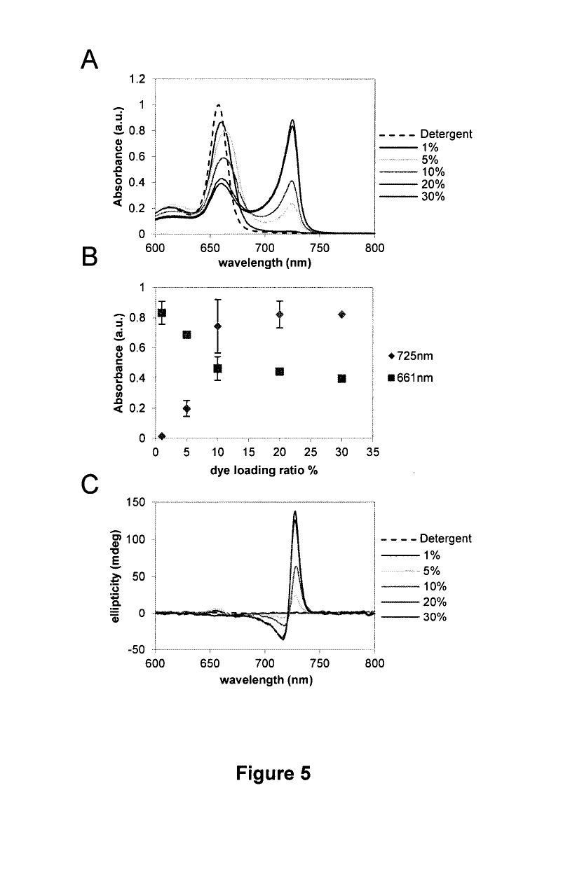

To further understand the factors governing the aggregation of Zn-MeO-chlorin lipid in nanovesicle membranes, we systematically titrated the Zn-MeO-chlorin lipid amount loaded (1-30 mol % of total lipid) into nanovesicle formulations (FIG. 5). As the dye was titrated from 1 to 20%, an increase in absorption was observed with a concomitant decrease in monomeric dye absorption (FIG. 5, A & B). Following a similar trend, CD spectra also showed an increase in observed Cotton effect at the Q_{y} band with higher dye loading (FIG. 5, C). Interestingly, neither UV/Vis spectra nor CD spectra significantly changed beyond 20% dye loading. Considering that Zn-MeO-chlorin can form n stacks due to the π-π interaction and metal coordination bonds¹³, it is possible these supramolecular aggregates could impart a degree of membrane tension on the lipid bilayer, thus imposing an upper limit to the size of the aggregate formed within a nanovesicle of defined size (FIG. 5 & Table S1). Further work will be required to investigate this observation in greater detail.

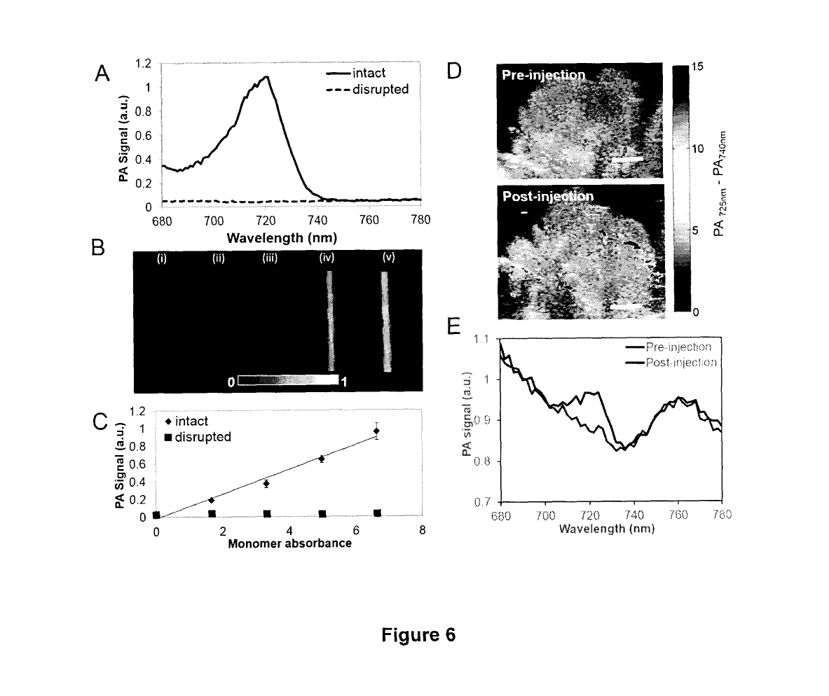

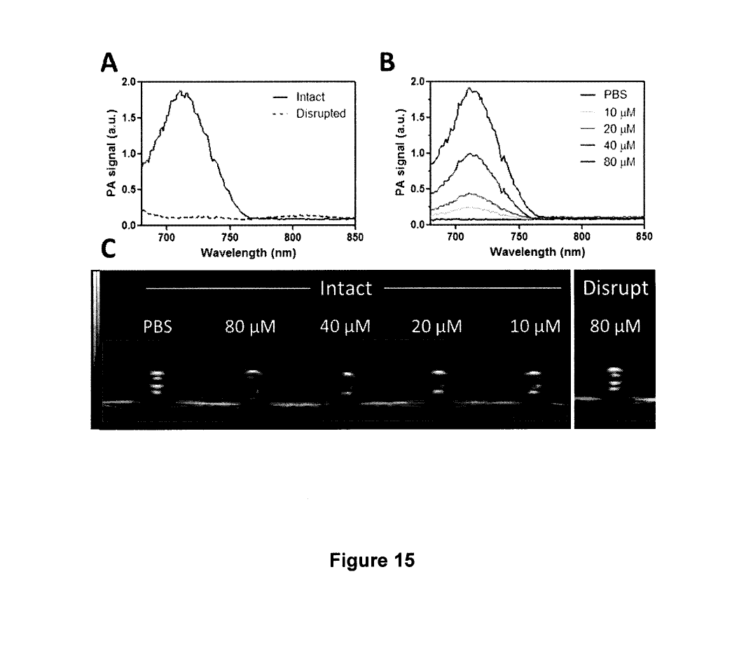

Next, we investigated the possible biomedical applications of Zn-MeO-chlorin lipid nanovesicles. Since 98% of fluorescence was quenched when the nanovesicles were intact (see FIG. 15), we postulated that Zn-MeO-chlorin lipid nanovesicles would possess a high efficiency of photothermal conversion. In addition to this, Zn-MeO-chlorin lipid nanovesicles display an absorption maxima in the near-infrared tissue optical window (700-900 nm) and a narrow full width at half-maximum (FWHM) of 21 nm. These features may allow for facile spectral unmixing from endogenous absorbers (hemoglobin, deoxyhemoglobin, melanin, etc.). We hypothesized that Zn-MeO-chlorin lipid embedded in nanovesicles could be used as contrast agents for PA imaging. A wavelength scan of PA value from 680 nm to 820 nm had a peak at 725 nm for intact particles and no signal for disrupted particles, which corresponded well with its absorption spectra (FIG. 4, A). The PA value with excitation at 725 nm exhibited a concentration-dependent increase in signal. However, once particles were disrupted with detergent, the signal was eliminated (FIG. 6, B & C). This effect could be explained by an increase in the dye fluorescence of the unquenched state and leads to a decrease in the propensity for relaxation through vibrational relaxation, the physical phenomenon responsible for the signal observed in PA imaging.

Lastly, as a proof-of-concept experiment, we tested whether the 20% Zn-MeO-chlorin-lipid nanovesicles could be detected within biological tissues. We utilized the chemically-induced hamster cheek pouch oral carcinoma for this model as it closely follows the events of oral carcinoma development and the chemically-induced tumor is accessible using the PA transducer. The tumor-bearing animal was anaesthetized with ketamine and the tumor was scanned with the PA transducer prior to the start of the experiment. Zn-MeO-chlorin lipid nanovesicles were administered intravenously following pre-scan and the signal in the tumor was monitored using photoacoustic imaging. A difference in signal contrast could be observed when comparing the pre-scan (top; FIG. 6, D) and the 5 min post-injection images (bottom; FIG. 6, D). Contrast enhancement was found to be focused at several loci throughout the tumor and could represent the signal originating from large blood vessels where the signal is expected to localize. A PA spectrum generated from the averaging of PA intensity and dividing by the area of the interest showed a slight increase of PA signal enhancement at 725 nm when compared to the pre-injection image (FIG. 6, E). These results demonstrate that these ordered aggregates within the lipid nanovesicles were detectable by PA imaging after intravenous injection.

For comparison of structure dependent aggregation of chlorin derivatives in the hydrophobic core of a lipoprotein nanoparticle, we have currently studied two chlorin analogs: Zn-vinyl-chlorin oleylamine and Zn—OH chlorin oleylamine (FIG. 11-13 chemical characterization). We will soon explore three more chlorin analogs, which include the following: OH-chlorin oleylamine without central zinc insertion, Zn—OH chlorin acid without the oleylamine conjugation, and Zn-MeO-chlorin oleylamine.

These dyes were combined with 1,2-dimyristoyl-sn-glycero-3-phosphocholine (DMPC) and formulated using bath sonication and Bioruptor sonication methods. R4F peptide was added for size modulation of the lipoprotein, which ranges from 10-20 nm in diameter.

The optical properties of lipoprotein nanoparticles loaded with 10:1 mol % DMPC lipid: Zn-vinyl-chlorin oleylamine or Zn—OH-chlorin oleylamine were investigated by UV/visible spectrophotometry. Zn-vinyl-chlorin oleylamine did not demonstrate appreciable shift in optical absorption with only 8 nm Q_{y}-band wavelength shifts between the monomeric form in methanol compared to the intact particle in PBS (FIG. 10, FIG. 14). Zn—OH-chlorin oleylamine encapsulation into the lipoprotein nanoparticle induced a 61 nm bathochromic shift in the lowest energy absorption band (Qy) compared to the absorption in its monomeric state in methanol.

Next, we investigated the possible biomedical applications of Zn—OH-chlorin lipoprotein nanoparticles for PA imaging. A wavelength scan of PA value from 680 nm to 850 nm had a peak at 715 nm for intact particles and no signal for disrupted particles, which corresponded well with its absorption spectra (FIG. 15, A). The PA value with excitation at 715 nm exhibited a concentration-dependent increase in signal (FIG. 15 B, C). This effect could be explained, like above with the Zn-MeO-chlorin lipid nanovesicles, by an increase in the dye fluorescence of the unquenched state and leads to a decrease in the propensity for relaxation through vibrational relaxation, the physical phenomenon responsible for the signal observed in PA imaging.

In summary, modifications of chlorin derivatives with a 3¹-methoxy group and insertion of zinc into core of the molecule can lead to template-induced ordered aggregation within the membrane of lipid nanovesicles and that both modifications are required for the formation of ordered aggregates. Furthermore, the increased red-shift and absorption of the lipid conjugated Zn-MeO-chlorin lipid versus the Zn-MeO-chlorin acid suggests that lipid conjugation promotes formation of more highly ordered aggregates possibly due to the aligning environment of the lipid bilayer. Lipid nanovesicles formed with Zn-MeO-chlorin lipid can be used as a PA imaging contrast agent with narrow NIR absorption band, which is favorable for spectral un-mixing in both tube phantoms and within the tumor of a spontaneously generated hamster oral carcinoma model.

Considering the advantages for using lipid building blocks to form a variety of biocompatible nanostructures such as liposomes, nanodiscs²⁶, micelles and microbubbles²⁷, and ongoing interest in using these amphiphiles as drug delivery carriers¹⁸, the findings in this paper may serve as a useful way in which to incorporate PA functionality in nano-agents for bioimaging and therapeutic applications.

Hydrophobic cargo core-loading of chlorin derivatives was enabled by replacing the phospholipid with a more lipophilic oleylamine attachment. The 3¹-OH group substitution to the chlorin-oleylamine was sufficient for an aggregation-induced bathochromic shift of the Q_{y}-band due to the additional proton capable of hydrogen bonding with the 13¹ keto group of a different monomer unit.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCES

1. Beatty, J. T.; Overmann, J.; Lince, M. T.; Manske, A. K.; Lang, A. S; Blankenship, R. E.; Van Dover, C. L.; Martinson, T. A.; Plumley, F. G. An Obligately Photosynthetic Bacterial Anaerobe from a Deep-Sea Hydrothermal Vent. Proc. Natl. Acad. Sci. U.S.A. 2005, 102, 9306-9310.
2. Manske, A. K.; Glaeser, J.; Kuypers, M. M. M.; Overmann, J. Physiology and Phylogeny of Green Sulfur Bacteria Forming a Monospecific Phototrophic Assemblage at a Depth of 100 Meters in the Black Sea. Appl. Env. Microbiol. 2005, 71, 8049-8060.
3. Kouyianou, K.; De Bock, P.-J.; Muiller, S. A.; Nikolaki, A.; Rizos, A.; Krzyinek, V.; Aktoudianaki, A.; Vandekerckhove, J.; Engel, A.; Gevaert, K., et al. The Chlorosome of Chlorobaculum Tepidum: Size, Mass and Protein Composition Revealed by Electron Microscopy, Dynamic Light Scattering and Mass Spectrometry-Driven Proteomics. Proteomics 2011, 11, 2867-2880.
4. Oostergetel, G. T.; van Amerongen, H.; Boekema, E. J. The Chlorosome: A Prototype for Efficient Light Harvesting in Photosynthesis. Photosynth. Res. 2010, 104, 245-255.
5. Ps̆enc̆ik, J.; Ikonen, T. P.; Laurinmaki, P.; Merckel, M. C.; Butcher, S. J.; Serimaa, R. E.; Tuma, R. Lamellar Organization of Pigments in Chlorosomes, the Light Harvesting Complexes of Green Photosynthetic Bacteria. Biophys. J. 2004, 87, 1165-1172.
6. Balaban, T. S.; Holzwarth, A. R.; Schaffner, K.; Boender, G.-J.; de Groot, H. J. M. Cp-Mas 13c-Nmr Dipolar Correlation Spectroscopy of 13c-Enriched Chlorosomes and Isolated Bacteriochlorophyll C Aggregates of Chlorobium Tepidum: The Self-Organization of Pigments Is the Main Structural Feature of Chlorosomes. Biochemistry 1995, 34, 15259-15266.
7. Smith, K. M.; Kehres, L. A.; Fajer, J. Aggregation of the Bacteriochlorophylls C, D, and E. Models for the Antenna Chlorophylls of Green and Brown Photosynthetic Bacteria. J. Am. Chem. Soc. 1983, 105, 1387-1389.
8. Brune, D. C.; Nozawa, T.; Blankenship, R. E. Antenna Organization in Green Photosynthetic Bacteria. 1. Oligomeric Bacteriochlorophyll C as a Model for the 740 Nm Absorbing Bacteriochlorophyll C in Chloroflexus Aurantiacus Chlorosomes. Biochemistry 1987, 26, 8644-8652.
9. Balaban, T. S. Self-Assembling Porphyrins and Chlorins as Synthetic Mimics of the Chlorosomal Bacteriochlorophylls. In Handbook of Porphyrin Science with Applications to Chemistry, Physics, Materials Science, Engineering, Biology and Medicine, Kadish, K. M.; Smith, K. M.; Guilard, R., Eds. World Scientific Publishing Co. Pte. Ltd.: Singapore, 2010; Vol. 1, pp 221-306.
10. Balaban, T. S.; Holzwarth, A. R.; Schaffner, K. Circular Dichroism Study on the Diastereoselective Self-Assembly of Bacteriochlorophyll Cs. J. Mol. Struct. 1995, 349, 183-186.
11. Ganapathy, S.; Sengupta, S.; Wawrzyniak, P. K.; Huber, V.; Buda, F.; Baumeister, U.; WOrthner, F.; de Groot, H. J. M. Zinc Chlorins for Artificial Light-Harvesting Self-Assemble into Antiparallel Stacks Forming a Microcrystalline Solid-State Material. Proc. Natl. Acad. Sci. U.S.A. 2009, 106, 11472-11477.
12. Miyatake, T.; Tanigawa, S.; Kato, S.; Tamiaki, H. Aqueous Self-Aggregates of Amphiphilic Zinc 31-Hydroxy- and 31-Methoxy-Chlorins for Supramolecular Light-Harvesting Systems. Tetrahedron Lett. 2007, 48, 2251-2254.
13. Huber, V.; Lysetska, M.; Wirthner, F. Self-Assembled Single- and Double-Stack Pi-Aggregates of Chlorophyll Derivatives on Highly Ordered Pyrolytic Graphite. Small 2007, 3, 1007-1014.
14. Ng, K. K.; Shakiba, M.; Huynh, E.; Weersink, R. A.; Roxin, A.; Wilson, B. C.; Zheng, G. Stimuli-Responsive Photoacoustic Nanoswitch for in Vivo Sensing Applications. ACS Nano 2014, 8, 8363-8373.
15. Zhang, D.; Zhao, Y.-X.; Qiao, Z.-Y.; Mayerhöffer, U.; Spenst, P.; Li, X.-J.; Wiurthner, F.; Wang, H. Nano-Confined Squaraine Dye Assemblies: New Photoacoustic and near-Infrared Fluorescence Dual-Modular Imaging Probes in Vivo. Bioconj. Chem. 2014, 25, 2021-2029.
16. Miyatake, T.; Tamiaki, H. Self-Assembly of Synthetic Zinc Chlorins in Aqueous Microheterogeneous Media to an Artificial Supramolecular Light-Harvesting Device. Helv. Chim. Acta 1999, 82, 797-810.
17. Allen, T. M.; Cullis, P. R. Drug Delivery Systems: Entering the Mainstream. Science 2004, 303, 1818-1822.
18. Allen, T. M.; Cullis, P. R. Liposomal Drug Delivery Systems: From Concept to Clinical Applications. Adv. Drug. Deliv. Rev. 2013, 65, 36-48.
19. Cui, L.; Tokarz, D.; Cisek, R.; Ng, K. K.; Wang, F.; Chen, J.; Barzda, V.; Zheng, G. Organized Aggregation of Porphyrins in Lipid Bilayer for Third Harmonic Generation Microscopy. Angew. Chem. Int. Ed. 2015.
20. Ng, K. K.; Zheng, G. Molecular Interactions in Organic Nanoparticles for Phototheranostic Applications. Chem. Rev. 2015.
21. Huber, V.; Sengupta, S.; Wiurthner, F. Structure-Property Relationships for Self-Assembled Zinc Chlorin Light-Harvesting Dye Aggregates. Chemistry 2008, 14, 7791-7807.
22. Miyatake, T.; Tamiaki, H. Self-Aggregates of Natural Chlorophylls and Their Synthetic Analogues in Aqueous Media for Making Light-Harvesting Systems. Coord. Chem. Rev. 2010, 254, 2593-2602.
23. MacDonald, R. C.; MacDonald, R. I.; Menco, B. P. M.; Takeshita, K.; Subbarao, N. K.; Hu, L.-r. Small-Volume Extrusion Apparatus for Preparation of Large, Unilamellar Vesicles. Biochim. Biophys. Acta, Biomembr. 1991, 1061, 297-303.
24. Barzda, V.; Mustardy, L.; Garab, G. Size Dependency of Circular Dichroism in Macroaggregates of Photosynthetic Pigment-Protein Complexes. Biochemistry 1994, 33, 10837-10841.
25. Wuirthner, F.; Kaiser, T. E.; Saha-Möller, C. R. J-Aggregates: From Serendipitous Discovery to Supramolecular Engineering of Functional Dye Materials. Angew. Chem. Int. Ed. 2011, 50, 3376-3410.
26. Ng, K. K.; Lovell, J. F.; Vedadi, A.; Hajian, T.; Zheng, G. Self-Assembled Porphyrin Nanodiscs with Structure-Dependent Activation for Phototherapy and Photodiagnostic Applications. ACS Nano 2013, 7, 3484-3490.
27. Huynh, E.; Leung, B., Y. C.; Helfield, B. L.; Shakiba, M.; Gandier, J.-A.; Jin, C. S.; Master, E. R.; Wilson, B. C.; Goertz, D. E.; Zheng, G. In Situ Conversion of Porphyrin Microbubbles to Nanoparticles for Multimodality Imaging. Nat. Nano. 2015, 10, 325-332.
28. Lovell, J. F.; Jin, C. S.; Huynh, E.; Jin, H.; Kim, C.; Rubinstein, J. L.; Chan, W. C.; Cao, W.; Wang, L. V.; Zheng, G. Porphysome Nanovesicles Generated by Porphyrin Bilayers for Use as Multimodal Biophotonic Contrast Agents. Nat. Mater. 2011, 10, 324-332.
29. Pallenberg, A. J.; Dobhal, M. P.; Pandey, R. K. Efficient Synthesis of Pyropheophorbide-a and Its Derivatives. Org. Process Res. Dev. 2004, 8, 287-290.
30. Tamiaki, H.; Amakawa, M.; Shimono, Y.; Tanikaga, R.; Holzwarth, A. R.; Schaffner, K. Synthetic Zinc and Magnesium Chlorin Aggregates as Models for SupramolecularAntenna Complexes in Chlorosomes of Green Photosynthetic Bacteria. Photochem. Photobiol. 1996, 63, 92-99.

1. A nanovesicle comprising a monolayer surrounding a hydrophobic core, the monolayer comprising phospholipid and the hydrophobic core comprising porphyrin-lipid conjugate, the porphyrin-lipid conjugate comprising one porphyrin, porphyrin derivative or porphyrin analog covalently bonded to a lipid, wherein
the lipid is an unsaturated or branched fatty acid that anchors the porphyrin-lipid conjugate to the monolayer;
the porphyrin, porphyrin derivative or porphyrin analog comprises a CH(R¹)—O—R² group covalently bonded to a carbon on a porphyrin ring of the porphyrin, porphyrin derivative or porphyrin analog, wherein R¹ and R² are independently H or a C₁₋₄ alkane; and
the porphyrin, porphyrin derivative or porphyrin analog comprises a metal chelated therein.

2. The nanovesicle of claim 1, wherein the lipid and CH(R¹)—O—R² group are bonded to separate pyrrole rings on the porphyrin ring.

3. The nanovesicle of claim 2, wherein the lipid and CH(R¹)—O—R² group are bonded to adjacent pyrrole rings on the porphyrin ring.

4. The nanovesicle of claim 1, wherein the CH(R¹)—O—R² group is bonded to the carbon at position 3 of the porphyrin ring.

5. The nanovesicle of claim 1, wherein R¹ and R² are independently methyl or ethyl, preferably both methyl.

6. The nanovesicle of claim 5, wherein R¹ and R² are both methyl.

7. The nanovesicle of claim 1, wherein the metal is Mg, Mn, Fe, Ni, Zn, Cu, Co or Pd, preferably Fe, Zn, Cu, Co, and Pd.

8. The nanovesicle of claim 7, wherein the metal is Zn or Pd.

9. The nanovesicle of claim 1, wherein said porphyrin-lipid conjugate comprises at least one oleate moiety, cholesterol oleate moiety or phytol moiety.

10. (canceled)

11. The nanovesicle of claim 1, wherein the fatty acid is bonded to the carbon at position 7, 10, 17 or 20 of the porphyrin ring.

12. (canceled)

13. The nanovesicle of claim 1, wherein the porphyrin, porphyrin derivative or porphyrin analog in the porphyrin-lipid conjugate is selected from the group consisting of hematoporphyrin, protoporphyrin, tetraphenylporphyrin, a pyropheophorbide, a bacteriochlorophyll, chlorophyll a, a benzoporphyrin derivative, a tetrahydroxyphenyl chlorin, a purpurin, a benzochlorin, a naphthochlorins, a verdin, a rhodin, a keto chlorin, an azachlorin, a bacteriochlorin, a tolyporphyrin, a benzobacteriochlorin, an expanded porphyrin and a porphyrin isomer.

14. The nanovesicle of claim 13, wherein the expanded porphyrin is a texaphyrin, a sapphyrin or a hexaphyrin and the porphyrin isomer is a porphycene, an inverted porphyrin, a phthalocyanine, or a naphthalocyanine.

15. The nanovesicle of claim 13, wherein the porphyrin, porphyrin derivative or porphyrin analog is pyropheophorbide-a chlorin.

16. The nanovesicle of claim 1, wherein the porphyrin, porphyrin derivative or porphyrin analog further comprises a ketone group bonded to a carbon on the porphyrin ring.

17.-18. (canceled)

19. The nanovesicle of claim 1, wherein the monolayer further comprises at least one peptide incorporated therein, the peptide selected from the group consisting of Class A, H, L and M amphipathic α-helices, fragments thereof, and peptides comprising a reversed peptide sequence of said Class A, H, L and M amphipathic α-helices or fragments thereof.

20. (canceled)

21. The nanovesicle of claim 19, wherein the peptide is a peptide of an apolipoprotein or an apolipoprotein mimetic.

22.-23. (canceled)

24. The nanovesicle of claim 1, wherein the phospholipid is selected from the group consisting of 1,2-dipalmitoyl-sn-glycero-3-phosphatidic acid (DPPA), 1,2-dipalmitoyl-sn-glycero-3-phosphatidylcholine (DPPC), 1,2-distearoyl-sn-glycero-3-phosphocholine (DSPC), 1,2-dimyristoyl-sn-glycero-3-phosphocholine (DMPC), 1,2-dibehenoyl-sn-glycero-3-phosphocholine (DBPC), 1,2-diarachidoyl-sn-glycero-3-phosphatidylcholine (DAPC), 1,2-dilignoceroyl-sn-glycero-3-phosphatidylcholine(DLgPC), 1,2-dipalmitoyl-sn-glycero-3-[phosphor-rac-(1-glycerol)] (DPPG) and combinations thereof.

25. The nanovesicle of claim 1, further comprising a PEG phospholipid.

26. The nanovesicle of claim 1, wherein the molar % of porphyrin-lipid conjugate to phospholipid is up to 70%.

27. (canceled)

28. The nanovesicle of claim 26, wherein the molar % of porphyrin-lipid conjugate to phospholipid is about 30%.

29. The nanovesicle of claim 26, wherein the molar % of porphyrin-lipid conjugate to phospholipid is about 20%.

30. The nanovesicle of claim 1, wherein the nanovesicle is substantially spherical and about 10-50 nm in diameter.

31. The nanovesicle of claim 1, wherein the nanovesicle exhibits a bathochromic shift towards the infrared spectrum.

32. The nanovesicle of claim 31, wherein the bathochromic shift is at least 40 nm-80 nm.

33. The nanovesicle of claim 1, further comprising a targeting molecule.

34. (canceled)

35. A method of performing photothermal therapy on a target area in a subject comprising:
a. providing the nanovesicle of claim 1;
b. administering the nanovesicle to the subject; and
c. irradiating the nanovesicle at the target area with a wavelength of light, wherein the wavelength of light increases the temperature of nanovesicle.

36. (canceled)

37. A method of imaging a target area in a subject, comprising
a. providing the nanovesicle of claim 1;
b. administering the nanovesicle to the subject; and
c. measuring and/or detecting the fluorescence at the target area.

38. A method of performing photodynamic therapy at a target area in a subject, comprising:
a. providing the nanovesicle of claim 1;
b. administering the nanovesicle to the subject;
c. allowing the porphyrin-lipid conjugate to disassociate from the nanovesicle at the target area; and
d. irradiating the target area with a wavelength of light, wherein the wavelength of light activates the nanovesicle to generate singlet oxygen.

39.-44. (canceled)

45. The method of claim 38, wherein the target area is a tumour.

46.-49. (canceled)
